(19) United States
(12) Patent Application Publication
(10) Pub. No.: US 2026/0062739 A1
(43) Pub. Date: Mar. 5, 2026

(54) FIELD-DEPLOYABLE FLUIDIC REACTOR

(71) Applicant: Helical Solutions LLC, Buckley, WA (US)

(72) Inventor: Tyler Boies, Buckley, WA (US)

(21) Appl. No.: 19/319,473

(22) Filed: Sep. 4, 2025

Related U.S. Application Data
(60) Provisional application No. 63/690,742, filed on Sep. 4, 2024.

(51) Int. Cl. C12Q 1/6844; C12N 9/22; C12N 15/113; C12Q 1/34; C12Q 1/6806

(52) U.S. Cl. CPC C12Q 1/6844; C12N 9/226; C12N 15/113; C12Q 1/34; C12Q 1/6806; C12Y 302/01004; C12Y 304/00; C12N 2310/20

(57) ABSTRACT

This disclosure describes techniques for detecting genetic material in a sample using a fluidic reactor device. The fluidic reactor device may receive a fluid containing genetic material to be processed through a filter component. The fluidic reactor device may react an enzyme with the fluid at a first component to expel genetic material from the filter component. The fluidic reactor device may also react another enzyme with the genetic material in order to generate extracellular genetic material, which may be amplified. CRISPR-Cas13 techniques may be performed by the fluidic reactor device on the amplified genetic material in order to generate fluorescence based on the presence of the amplified extracellular genetic material.

### RELATED APPLICATIONS

This application claims priority to U.S. Provisional Application No. 63/690,742, filed Sep. 4, 2024 entitled “Field-Deployable Fluidic Reactor,” the entirety of which is herein incorporated by reference.

### BACKGROUND

The presence of genetic material (e.g., DNA, RNA, etc.) in the environment, such as in water systems, air systems, wastewater, etc., can provide an abundance of information about the environment. For example, genetic material in the environment can be used to track species populations, the presence of a virus in wastewater, and/or the like. Typically, to gather such genetic material and eventually detect its presence requires a laborious process, both physically and temporally. For example, an agent (e.g., researcher, biologist, etc.) may be required to go into the field, collect samples from the environment, hand filter the sample, and bring the samples back to a laboratory. Even further, the samples are historically processed using Quantitative Polymerase Chain Reaction (qPCR), also referred to as Real-Time PCR. The qPCR process may detect and quantify target genetic material by denaturation, primer annealing, extension, and quantification of the target genetic material. However, qPCR itself may also be a laborious process, and typically requires the use of a stationary thermal cycler. Accordingly, current testing methods are difficult, time-consuming, and inflexible.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an example fluidic reactor device and various components described herein, according to at least some examples.

FIGS. 2A-2C illustrate an example filter component of the fluidic reactor device, according to at least some examples.

FIG. 3 illustrates an example adapter that may be coupled to the filter component of the fluidic reactor device, according to at least some examples.

FIGS. 4A-4C illustrate example modular reactors, or pumps, which may be included in the fluidic reactor device, according to at least some examples.

FIGS. 5A and 5B illustrate an example light detection component that may be included in the fluidic reactor device, according to at least some examples.

FIG. 6 illustrates an example computing component and display that may be included in the fluidic reactor device, according to at least some examples.

FIGS. 7A and 7B illustrate another example modular reactor, or pump, which may be included in the fluidic reactor device, according to at least some examples.

FIG. 8 illustrates another example module reactor, or pump, which may be included in the fluidic reactor device, according to at least some examples.

FIG. 9 illustrates an example sampling component that may be included in, or work in combination with, the fluidic reactor device, according to at least some examples.

FIG. 10 illustrates an example module reactor and light detection component that may be included in the fluidic reactor device and used in combination with the sampling component of FIG. 9, according to at least some examples.

FIGS. 11A and 11B illustrate additional examples of components to be included in the fluidic reactor device, according to at least some examples.

FIG. 12 illustrates a flow diagram of an example process for a fluidic reactor device to detect genetic material in a sample, according to at least some examples.

FIG. 13 illustrates a flow diagram of another example process for a fluidic reactor device to detect genetic material in a sample, according to at least some examples.

### DETAILED DESCRIPTION

This application is directed, at least in part, to a fluidic reactor device for detecting genetic material in a sample. The fluidic reactor device described herein may include a filter component. Additionally, or alternatively, the filter component may be associated with a fluid intake component. By way of example, and not limitation, the fluidic reactor device may be disposed in an environment, such as in or near a waterway, wastewater system, ventilation system, open air environment, and/or any other environmental feature where genetic material may be detected in a fluid (e.g., gas, liquid, etc.), semi-solid substrates (e.g., soil slurries, biofilms, etc.), and/or solid biological samples (e.g., tissue homogenates, swabs, etc.). The fluidic reactor device may be disposed in an environment for biosensing (e.g., detecting pathogens), stationary lab work (e.g., environmental monitoring), as a portable field unit field unit (e.g., in extreme conditions), and/or the like. In some examples, the fluid intake component may include, or work in combination with, a siphon, pump (e.g., peristaltic, diaphragm, centrifugal, etc.), vacuum, fan (e.g., blower, external pressure differentials such as an HVAC airflow, etc.), funnel, etc. to collect a fluid (e.g., liquid or gas) from the environment, where the fluid intake component may cause the fluid to be collected and disposed in the filter component of the fluidic reactor device. The fluid intake component or portion thereof may be actively driven (e.g., by a motor) to pull fluid into the fluidic reactor device, or may passively collect fluid using the flow of the fluid or other naturally occurring forces or conditions (flow of a waterway, wastewater system, siphon, flow of air through a ventilation duct, wind, etc.). Additionally, or alternatively, the fluid intake component may be associated with one or more adapters configured to aid in the collection of a fluid from the environment. For example, an adapter may be coupled to the fluid intake component such that the fluidic reactor device may be adapted to different scenarios, environments, etc. For example, the adapter may include a tank that is configured to store collected fluids and/or solvents for further processing of the collected fluids before an interaction with the filter component of the fluidic reactor device. In some examples, the fluid intake component may collect fluid continuously or periodically.

In some examples, the fluidic reactor device may be used in combination with an autonomous vehicle (e.g., drone, car, plane, airship, etc.) and/or platform (e.g., surface buoy) for the collection of target genetic material. By way of example, and not limitation, the fluidic reactor device may be coupled to an airborne drone and/or waterborne drone. For example, in instances where the fluidic reactor device is coupled to an airborne drone, the fluid reactor device may be configured to collect target genetic material from a fluid such as gaseous media (e.g., aerosolized particles in atmospheric air, enclosed spaces, etc.) using a fan. In some instances, a fan used for collecting target genetic material may be used in combination with a propulsion system of the airborne drone. Additionally, the pore size of one or more filters used by the fluidic reactor device for collecting gaseous media may be dependent on and/or optimized for environmental and/or drone conditions (e.g., filters with larger pore size to reduce drag in high-flow environments, filters with smaller pore size for higher sensitivity in low-flow environments). In another example, in instances where the fluidic reactor device is coupled to a waterborne drone, the fluid reactor device may be configured to collect target genetic material from a fluid such as water using a pump. In some instances, a pump used for collecting target genetic material may be positioned so as to avoid propulsion turbulence.

Once the fluid has been collected and disposed in the filter component, the fluid may subsequently be filtered through a filter. In some examples, the filter may comprise cellulose, though in other examples filters may additionally or alternatively include one or more other materials (e.g., polymers, ceramics, metals, etc.). The filter may be placed in the filter component via a filter feeder, where the filter feeder may store one or more filters to be loaded into the filter component. The filter may be configured to collect and/or attract genetic material (e.g., cells containing DNA and/or RNA) that is present in the fluid. For example, the filter may collect and/or attract the genetic material due to hydrogen bonding, hydrophobic interactions, and/or other types of bonding and/or intermolecular forces. Additionally, or alternatively, the filter component may include a spout for filtered fluid. In some examples, the spout may be configured to discharge the fluid once the fluid has been filtered through the filter of the filter component. Additionally, or alternatively, the filter component may include, or work in combination with, one or more gears, wherein the one or more gears are configured to direct filtered fluid into a removal channel, where the fluid is then discharged via the spout. For example, one of the gears may be actuated by a motor, such as a stepper motor. In some examples, another gear may be coupled to one or more blades, where the blades may direct the filtered fluid into the removal channel. Additionally, or alternatively, the filter component may include a platform with one or more filter cavities, such that multiple filters, with collected genetic material attached thereto, may be placed in the filter cavities. This way, one or more filters may be rotated and/or shifted for further processing. In instances where a gaseous fluid is being used for the detection of genetic material, the fluidic reactor device may also be configured to filter the gaseous fluid using a filter component, where the filter component may include a fan and ducting system, where a filter may be coupled to the ducting system for the collection of genetic material from a gaseous fluid pushed through the filter component by a an.

In some instances, the filter component may be coupled to one or more pumps (or “reactors”). For example, the filter component may be coupled to the one or more pumps via a tube. The pumps may comprise pressure-driven pumps. In some instances, one pump may be configured to generate a solution that is usable, when introduced with a filter, to expel the genetic material from the filter and into the solution. In some instances, the genetic material may be expelled from the filter and into the solution at the filter component. For example, when a cellulose filter is used, the solution may include an enzyme, such as cellulase, that catalyzes decomposition of the cellulose filter, and in turn, causes the genetic material collected by the cellulose filter (e.g., cells containing DNA and/or RNA) to be released into the solution. In the case of other types of filter materials (e.g., chitin, silica, polycarbonate, magnetic beads, and/or the like), other types of enzymes or techniques may be used to release the genetic material from the filter media. Example techniques for releasing the genetic material from the filter media may include mechanical disruption, chemical lysis, non-enzymatic lysis (e.g., acid-base hydrolysis), electroporation, etc. Additionally, or alternatively, the solution may include an enzyme, such as proteinase K, which catalyzes the break-down of proteins (e.g., proteins present in a cell membrane and/or a nuclear membrane) via the hydrolysis of peptide bonds. This way, genetic material that is contained within a nucleus of a cell may be released into the fluid and/or solution as extracellular genetic material.

In order to generate the cellulase solution and/or a proteinase-containing solution (and/or other enzyme-containing solutions for different filter types), the pump may include a capsule feeding component that may store one or more capsules of a lyophilized cellulase, lyophilized proteinase K, and/or another type of enzyme. In some examples, the one or more capsules may be configured to dissolve when interacted with fluid and/or solution. Further, the pump may use, or work in combination with, a motor, such as a stepper motor. In some examples, the capsule feeding component may be configured to deposit a capsule containing the lyophilized cellulase and/or proteinase K into a capsule housing that is coupled with the motor. Additionally, or alternatively, the pump and the motor may be coupled with a threaded rod, stock, and/or other linkage. This way, the motor may be configured to “push” the capsule into the pump such that the capsule interacts with a solution inside the pump, such as a fluid and/or solution from the tube, and in turn, causes the capsule to dissolve. Additionally, or alternatively, the pump may use, or work in combination with, an additional motor, such as a stepper motor. In some examples, the pump and the motor may be coupled with a magnet rod, which may contain one or more cavities for storing a magnet. Further, inside the pump may include a magnetic stir bar in proximity to the magnet, such that the magnet may cause the magnetic stir bar to spin. In some examples, the motor may actuate the spinning of the magnet, and thus cause the magnetic stir bar to spin. This way, when the capsule interacts with a solution inside the pump, the magnetic stir bar may aid in the dissolution of the capsule. Additionally, or alternatively, the pump may use, or work in combination with, a heating element disposed in, on, or adjacent to the pump or other reaction chamber to aid in the dissolution of the capsule and/or to subsequently denature an enzyme (e.g., proteinase K).

In one non-limiting example, a capsule containing a lyophilized cellulase enzyme may be dissolved in a solution at a first pump using the components and/or techniques described above. The cellulase solution may then be introduced, via a tube, to the filter component such that a cellulase filter containing genetic material may decomposed and/or degraded, and cause genetic material to be released in solution. Additionally, or alternatively, a capsule containing a lyophilized proteinase K enzyme may be dissolved in a solution at a second pump using the components and/or techniques described above. The proteinase K solution may then be introduced, via a tube, to the released genetic material in solution such that any membranes containing the genetic material may be broken-down, or lysed, and the remaining proteinase K enzyme is denatured via the application of heat. This way, the genetic material may be extracellular.

Additionally, or alternatively, the fluidic reactor device may include one or more additional pumps, or reactors, configured with the same and/or similar components as described above and enabled to perform the same and/or similar techniques as described above. For example, one or more additional pumps may be used to perform multiple instances of the same and/or similar test for redundancy purposes, checking accuracy, etc. Additionally, or alternatively, one or more additional pumps may be used to perform the same and/or similar techniques as described above with respect to different genetic targets (e.g., to detect DNA and/or RNA associated with different species, classifications, and/or the like). In other words, the fluidic reactor device may be modular and/or scalable. In some instances, the one or more pumps described above may be coupled to the one or more additional pumps via a tube. In some instances, one additional pump may be configured to perform techniques such as loop-mediated isothermal amplification (LAMP). For example, a capsule in a first additional pump may contain primers, polymerases, reagents, and/or other materials required for LAMP. As described above, the capsule may be loaded, or “pushed” into the first additional pump, and further processed using stirring and/or heating to dissolve the capsule in solution. Additionally, or alternatively, the genetic material, after being released from the filter and further processed using proteinase K, may be introduced to the solution at the first additional pump, where the solution may contain primers, polymerases, reagents, and/or other materials required for LAMP. Subsequently, the genetic material may be amplified using LAMP. It should be appreciated that other techniques may be used for isothermal amplification (e.g., nucleic acid sequence-based amplification (NASBA), helicase-dependent amplification (HDA), rolling circle amplification (RCA), strand displacement amplification (SDA), exponential amplification reaction, nicking enzyme amplification, signal-mediated amplification of RNA technology (SMART), and/or the like.

Additionally, or alternatively, a second additional pump may be configured to perform techniques such as the use of Clustered Regularly Interspaced Short Palindromic Repeats (CRISPR) systems with CRISPR-associated protein 12 (Cas12) and/or CRISPR-associated protein 13 (Cas13). For example, a capsule in the second additional pump may contain the targeted genetic material. For example, the capsule may contain guide RNA (gRNA) including a nucleotide spacer sequence indicative of the targeted genetic material (e.g., DNA and/or RNA). The capsule may also contain endonucleases (e.g., Cas12 and/or Cas13), reagents, and/or other materials required for CRISPR-Cas12 and/or CRISPR-Cas13 techniques. Additionally, or alternatively, the capsule may contain single-stranded genetic material (e.g., ssDNA and/or ssRNA). The single-stranded genetic material may contain, or be attached with, a probe, such as a charged particle and/or fluorophore. As described above, the capsule may be loaded, or “pushed” into the second additional pump, and further processed using stirring and/or heating to dissolve the capsule in solution. Additionally, or alternatively, the amplified genetic material may be introduced to the solution at the second additional pump, where the amplified genetic material may be further processed using CRISPR-Cas12 and/or CRISPR-Cas13. By way of example, and not limitation, if the amplified genetic material contains the sequence indicated by the gRNA, the Cas12 and/or Cas13 may be configured to cleave the genetic material (e.g., cleave the strands of DNA). Additionally, or alternatively, all local single-stranded genetic material present may be destroyed, such as the single-stranded genetic material attached with a probe. Accordingly, in response to the Cas12 and/or Cas13 finding the target sequence and cleaving the genetic material, a voltage change may occur (e.g., when the probe is a charged particle) and/or light may be emitted (e.g., when the probe is a fluorophore) due to the probe being cleaved from the single-stranded genetic material. In another example, if the amplified genetic material does not contain the sequence indicated by the gRNA, genetic material may not be cleaved and/or otherwise destroyed, and as such, no voltage change and/or light emittance may occur.

While the above system describes the use of multiple pumps, the same components and/or techniques may also be similarly used and/or performed using a singular pump. For example, the singular pump may contain one or more reaction components configured to receive one or more capsules, such as the capsules described above, and perform various reactions therein. For example, one reaction component may be configured to receive a capsule containing lyophilized cellulase enzyme, such that the cellulase solution, as described above, may be produced in the reaction component and configured to catalyze the decomposition of the filter at the filter component. In another example, one reaction component may be configured to receive a capsule containing lyophilized proteinase K, such that the proteinase-containing solution, as described above, may be produced in the reaction component and configured to lyse cellular membranes containing the genetic material. In another example, one reaction component may be configured to receive a capsule containing the materials and/or reagents required for LAMP techniques, such that the extracellular genetic material, as described above, may be amplified. In another example, one reaction component may be configured to receive a capsule containing the materials and/or reagents required for CRISPR-Cas12 and/or CRISPR-Cas13 techniques such that target genetic material, as described above, may be detected. The one or more reaction components may be coupled to the filter component and/or other components via a tube.

While the singular pump may use any of the components, techniques, and/or mechanisms described above with the multiple-pump system, the singular pump may include, but is not limited to, a capsule feeding component, platform, housing, motor(s), and/or gear(s) to perform the techniques described herein. For example, the capsule feeding component may store one or more capsules containing the materials and/or reagents as described above. Further, the pump may use, or work in combination with, a motor, such as a stepper motor. In some examples, the motor may be coupled to a capsule housing containing the one or more reaction components and a gear. The motor and gear may also be coupled to a platform, where the platform may be disposed between the capsule feeding component and the housing. Accordingly, the motor and gear may be configured to actuate and/or otherwise move the platform such that the one or more capsules may be deposited into the one or more reaction components, and techniques described above may be performed.

Additionally, or alternatively, the fluidic reactor device may include a light detection component. For example, the light detection component may be coupled to the singular pump system and/or the multiple pump system. In some examples, the light detection component may be coupled to the singular pump system and/or the multiple pump system via a tube, where a solution containing the genetic material after CRISPR-Cas12 and/or CRISPR-Cas13 has been performed may be received at the light detection component. While described herein as a light detection component, the light detection component may comprise a voltage detection component. For example, as described above, in response to the Cas12 and/or Cas13 finding the target sequence and cleaving the genetic material, a voltage change may occur (e.g., when the probe is a charged particle) and/or light may be emitted (e.g., when the probe is a fluorophore). For example, in instances where the probe is a fluorophore, the light detection component may be configured to expose the solution containing the genetic material to light (e.g., blue light and/or light with approximately 400 nanometers wavelength), and cause the emittance of light from the fluorophore (e.g., green light and/or light with approximately 550 nanometers wavelength). Accordingly, a binary result indicating the presence of the target genetic material in the originally collected fluid sample may be produced. For example, in instances where the probe is a fluorophore, if the target genetic material is not present in the originally collected fluid sample, there may be not emittance of light, or fluorescence, at the light detection component. As such, the binary result may indicate that the target genetic material is not present. In order to determine the binary result indicating the presence of the target genetic material or the absence of the target genetic material, the light detection component may be associated with a threshold fluorescence used to determine whether the target genetic material is present or absent. Target genetic material may include, or be associated with, a viral microbe, a prokaryotic organism, a eukaryotic organism, and/or the like. It is to be appreciated that other isothermal detection modalities may be used besides CRISPR-Cas12, CRISPR-Cas13, fluorescence, etc. For example, lateral flow assays, electrochemical sensors, nanoparticle aggregation, surface plasmon resonance, intercalating dyes, molecular beacons, toehold switch sensors, hybridization chain reaction (HCR), and/or the like may be used for detection techniques.

Additionally, or alternatively, the fluidic reactor device may include, or work in combination with, a computing system and/or user display. For example, the computing system may include processor(s) that may be implemented as appropriate in hardware, computer-executable instructions, firmware, and/or combinations thereof. Computer-executable instructions or firmware implementations of the processor(s) may include computer-executable and/or machine-executable instructions written in any suitable programming language to perform the various functions and/or techniques described. The computing system may also include memory configured to store program instructions that are loadable and executable on the processor(s), as well as data generated during the execution of these programs. The computing system may also include storage, such as either removable storage or non-removable storage including, but not limited to, magnetic storage, optical disks, and/or tape storage. The computing system may include one or more communication connections (e.g., radios, antennas, wired and/or wireless interfaces, etc.) configured for communication with a remote computing device (e.g., mobile device, laptop, computer, etc.) via one or more wired and/or wireless network interfaces. The computing system may be capable of establishing a communication session with a remote computing device and/or transmitting/receiving data from that remote computing device. The user display may include a communications interface that may include physical buttons (e.g., mechanical, resistive, capacitive, etc.), touch screens, input devices (e.g., keyboard, keypad, mouse, trackball, trackpad, stylus, cameras, microphones, etc.), or the like. The communications interface of the user display may also include communications with the computing system and/or the remote computing device to provide instructions to the fluidic reactor device. Additionally, or alternatively, the remote computing device may be configured to provide instructions to the fluidic reactor device via the computing system and/or the communications interface of the user display.

In some examples, the fluidic reactor device may include one or more power sources to provide energy to the fluidic reactor device and power operations, techniques, and/or mechanisms of the fluidic reactor device. The power sources may include primary sources of energy such as solar power, chemical energy, hydropower, and/or the like. Additionally, or alternatively, the power sources may include secondary energy sources, such as electricity.

As used herein, a processor, such as processor(s) and/or the processor(s) described with respect to the components of the system, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) and/or the processor(s) described with respect to the components of the system may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) and/or the processor(s) described with respect to the components of the system may possess its own local memory, which also may store program components, program data, and/or one or more operating systems.

The memory and/or the memory described with respect to the components of the system may include volatile and nonvolatile memory, removable and non-removable media implemented in any method or technology for storage of information, such as computer-readable instructions, data structures, program component, or other data. Such memory and/or the memory described with respect to the components of the system may include, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory and/or the memory described with respect to the components of the system may be implemented as computer-readable storage media (“CRSM”), which may be any available physical media accessible by the processor(s) and/or the processor(s) described with respect to the system to execute instructions stored on the memory and/or the memory described with respect to the components of the system. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s).

Further, functional components may be stored in the respective memories, or the same functionality may alternatively be implemented in hardware, firmware, application specific integrated circuits, field programmable gate arrays, or as a system on a chip (SoC). In addition, while not illustrated, each respective memory, such as memory and/or the memory described with respect to the components of the system, discussed herein may include at least one operating system (OS) component that is configured to manage hardware resource devices such as the network interface(s), the I/O devices of the respective apparatuses, and so forth, and provide various services to applications or components executing on the processors. Such OS component may implement a variant of the FreeBSD operating system as promulgated by the FreeBSD Project; other UNIX or UNIX-like variants; a variation of the Linux operating system as promulgated by Linus Torvalds; the FireOS operating system from Amazon.com Inc. of Seattle, Washington, USA; the Windows operating system from Microsoft Corporation of Redmond, Washington, USA; LynxOS as promulgated by Lynx Software Technologies, Inc. of San Jose, California; Operating System Embedded (Enca OSE) as promulgated by ENEA AB of Sweden; and so forth.

The network interface(s) and/or the network interface(s) described with respect to the components of the system may enable messages between the components and/or devices shown in environment and/or with one or more other polling systems, as well as other networked devices. Such network interface(s) and/or the network interface(s) described with respect to the components of the system may include one or more network interface controllers (NICs) or other types of transceiver devices to send and receive messages over the network.

For instance, each of the network interface(s) and/or the network interface(s) described with respect to the components of the system may include a personal area network (PAN) component to enable messages over one or more short-range wireless message channels. For instance, the PAN component may enable messages compliant with at least one of the following standards IEEE 802.15.4 (ZigBee), IEEE 802.15.1 (Bluetooth), IEEE 802.11 (WiFi), or any other PAN message protocol. Furthermore, each of the network interface(s) and/or the network interface(s) described with respect to the components of the system may include a wide area network (WAN) component to enable message over a wide area network. Additionally, or alternatively, each of the network interface(s) and/or the network interface(s) described with respect to the components of the system may include cellular networks.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

Additional details are described below with reference to several example embodiments.

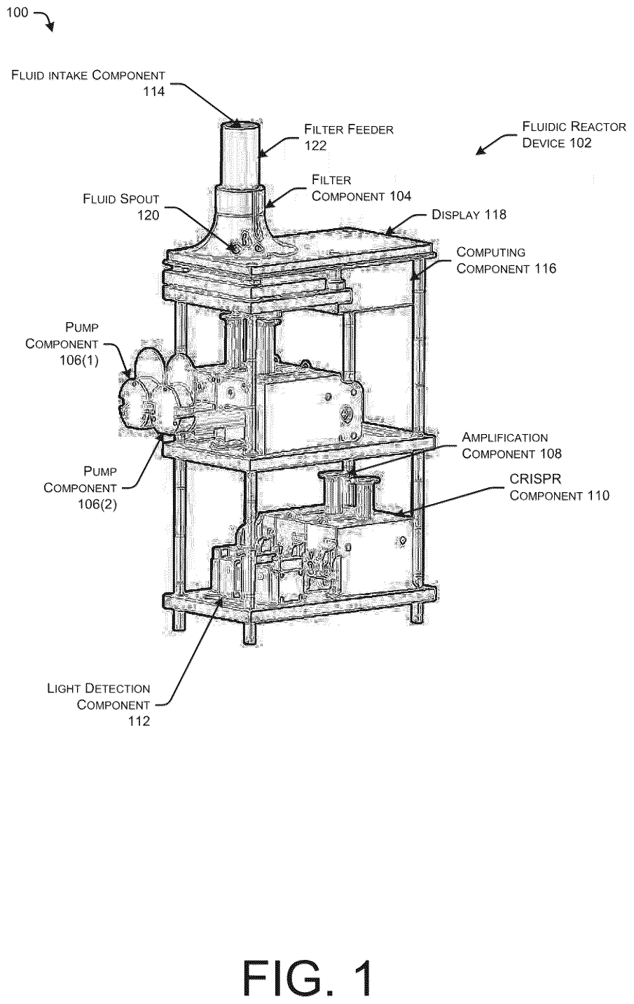

FIG. 1 illustrates an example 100 fluidic reactor device 102 and various components described herein, according to at least some examples. The fluidic reactor device 102 described herein may include a filter component 104. Additionally, or alternatively, the filter component 104 may be associated with a fluid intake component 114. By way of example, and not limitation, the fluidic reactor device 102 may be disposed in an environment, such as near a waterway, wastewater system, and/or any other environmental feature where genetic material may be detected in a fluid (e.g., gas, liquid, etc.). In some examples, the fluid intake component 114 may include, or work in combination with, a siphon, pump, vacuum, fan, funnel, etc. to collect a fluid from the environment, where the fluid intake component 114 may cause the fluid to be collected and disposed in the filter component 104 of the fluidic reactor device 102. The fluid intake component 114 or portion thereof may be actively driven (e.g., by a motor) to pull fluid into the reactor, or may passively collect fluid using the flow of the fluid or other naturally occurring forces or conditions (flow of a waterway, wastewater system, siphon, flow of air through a ventilation duct, wind, etc.). In some examples, the fluid intake component 114 may collect fluid continuously or periodically. Additionally, or alternatively, the filter component 104 may include a filter feeder 122. The filter feeder 122 may store one or more filters for use in the filter component 104. For example, once the fluid has been collected and disposed in the filter component 104, the fluid may subsequently be filtered through a filter. In some examples, the filter may comprise cellulose, though in other examples filters may additionally or alternatively include one or more other materials (e.g., polymers, ceramics, metals, etc.). The filter may be placed in the filter component 104 via the filter feeder 122, where the filter feeder 122 may store one or more filters to be loaded into the filter component 104. Additionally, or alternatively, the filter component 104 may include a fluid spout 120 for filtered fluid. In some examples, the fluid spout 120 may be configured to discharge the fluid once the fluid has been filtered through the filter of the filter component 104.

In some instances, the filter component 104 may be coupled to one or more pump components, such as pump component 106(1) and/or pump component 106(2). For example, the filter component 104 may be coupled to the pump component 106(1). In some instances, the pump component 106(1) may be configured to generate a solution that is usable, when introduced with a filter in the filter component 104, to expel the genetic material from the filter and into the solution. In some instances, the genetic material may be expelled from the filter and into the solution at the filter component 104. For example, in instances where the filter may be a cellulose filter, the solution may include an enzyme, such as cellulase, that catalyzes the decomposition of the cellulose filter, and in turn, causes the genetic material collected by the cellulose filter (e.g., cells) to be released into the solution. Additionally, or alternatively, the filter component 104 and/or the pump component 106(1) may be coupled to pump component 106(2). In some instances, the pump component 106(2) may be configured to generate a solution that may include an enzyme, such as proteinase K, which catalyzes the break-down of proteins (e.g., proteins present in a cell membrane and/or a nuclear membrane). This way, genetic material (e.g., DNA and/or RNA) expelled from the filter component 104 that is contained within a nucleus may be released into the solution as extracellular genetic material.

Additionally, or alternatively, the fluidic reactor device 102 may include one or more additional pump components, such as amplification component 108 and/or CRISPR component 110 configured with the same and/or similar components as described above and enabled to perform the same and/or similar techniques as described above. Although not illustrated, the pump components 106(1) and/or pump component 106(2) may be coupled to the amplification component 108 and/or CRISPR component 110 via a tube. In some instances, the amplification component 108 may be configured to perform techniques such as loop-mediated isothermal amplification (LAMP). For example, the genetic material may be amplified using LAMP. In some examples, the amplification component 108 may be coupled with the CRISPR component 110, where the two components may run simultaneously. Additionally, or alternatively, the CRISPR component 110 may be configured to perform techniques such as the use of Clustered Regularly Interspaced Short Palindromic Repeats (CRISPR) systems with CRISPR-associated protein 12 (Cas12) and/or CRISPR-associated protein 13 (Cas13). For example, in response to the Cas12 and/or Cas13 finding the target sequence in the amplified genetic material, a voltage change may occur (e.g., when the probe is a charged particle) and/or light may be emitted (e.g., when the probe is a fluorophore). The voltage change and/or the light emittance may indicate the presence of a target genetic material.

Additionally, or alternatively, the fluidic reactor device 102 may include a light detection component 112. For example, the light detection component 112 may be coupled to the CRISPR component 110 via a tube, where a solution containing the genetic material after CRISPR-Cas12 and/or CRISPR-Cas13 has been performed may be received at the light detection component 112. While described herein as a light detection component 112, the light detection component 112 may comprise a voltage detection component. For example, as described above, in response to the Cas12 and/or Cas13 finding the target sequence and cleaving the genetic material, a light may be emitted (e.g., when the probe is a fluorophore). For example, in instances where the probe is a fluorophore, the light detection component 112 may be configured to expose the solution containing the genetic material to light (e.g., blue light and/or light with approximately 400 nanometers wavelength), and cause the emittance of light from the fluorophore (e.g., green light and/or light with approximately 550 nanometers wavelength). Accordingly, a binary result indicating the presence of the target genetic material in the originally collected fluid sample may be produced. For example, in instances where the probe is a fluorophore, if the target genetic material is not present in the originally collected fluid sample, there may be not emittance of light at the light detection component 112. As such, the binary result may indicate that the target genetic material is not present.

Additionally, or alternatively, the fluidic reactor device 102 may include, or work in combination with, a computing component 116 and/or display 118. The computing component 116 may be capable of establishing a communication session with a remote computing device and/or transmitting/receiving data from that remote computing device. The display 118 may include a communications interface that may include physical buttons (e.g., mechanical, resistive, capacitive, etc.). touch screens, etc. The communications interface of the display 118 may also include communications with the computing component 116 and/or the remote computing device to provide instructions to the fluidic reactor device 102. Additionally, or alternatively, the remote computing device may be configured to provide instructions to the fluidic reactor device 102 via the computing component 116 and/or the communications interface of the display 118.

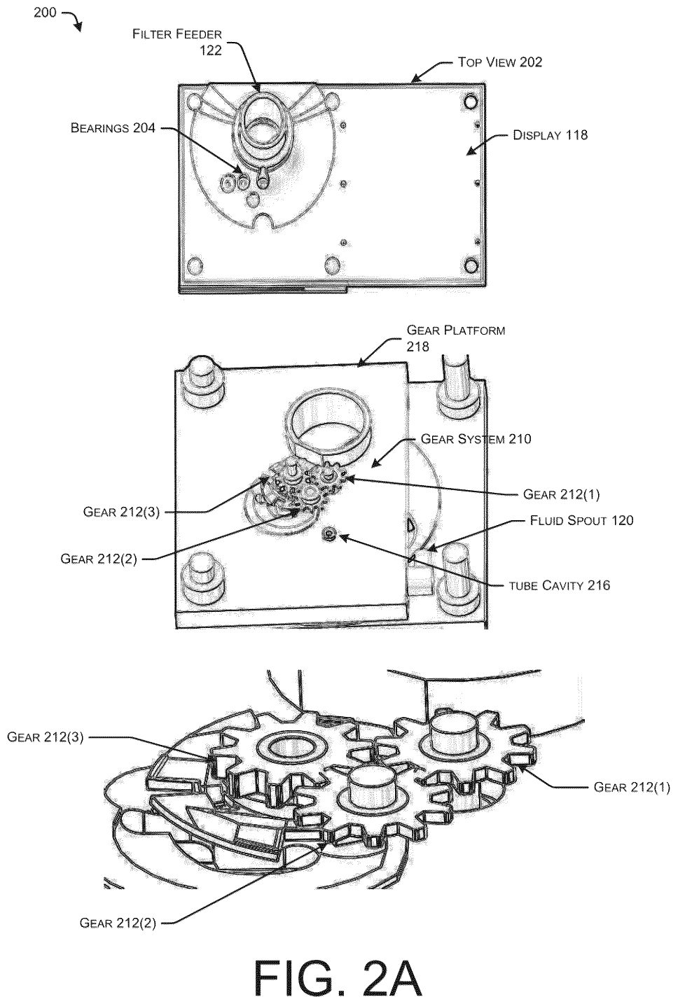

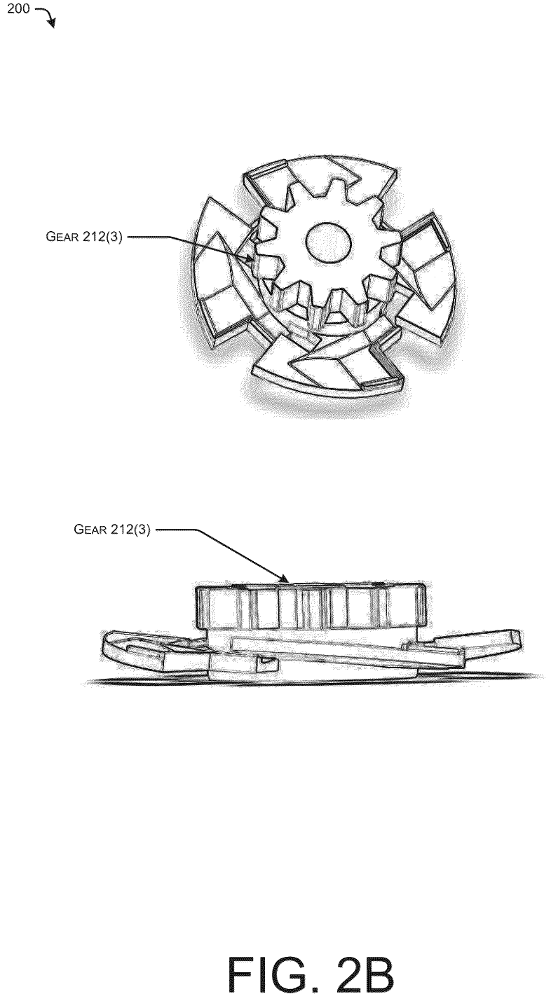

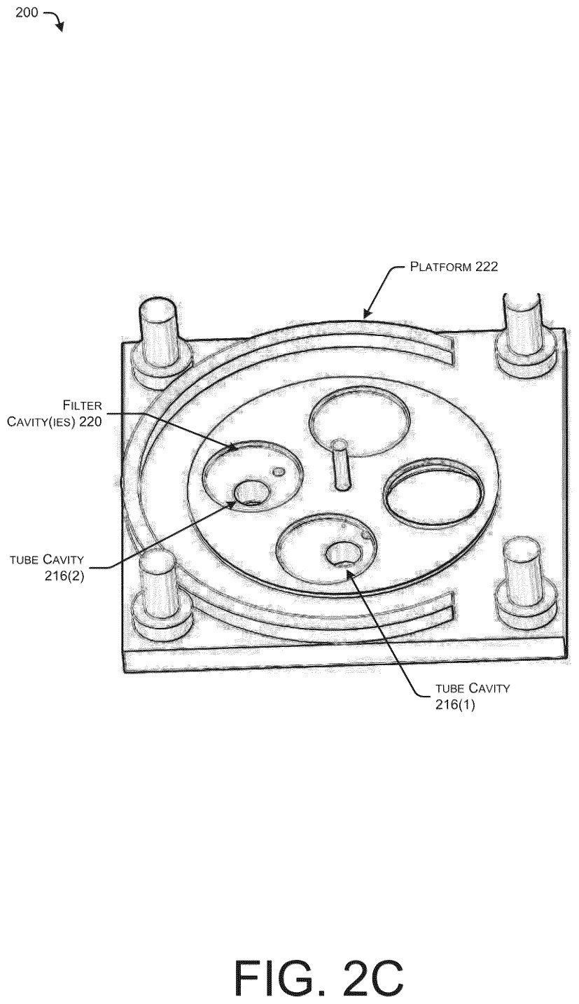

FIGS. 2A-2C illustrate an example 200 filter component, such as a filter component 104, of the fluidic reactor device, according to at least some examples. As illustrated in FIG. 2A, a top view 202 of a fluidic reactor device illustrates the filter feeder 122, bearings 204, and display 118. In some examples, the bearings 204 may be configured to connect multiple levels, or platforms, of the filter component. For example, below the top view 202 may include a gear platform 218 with gear system 210. In some examples, the gear system 210 may be configured to direct filtered fluid into a removal channel, where the fluid is then discharged via fluid spout 120. Additionally, or alternatively, the gear platform 218 may include a tube cavity 216, where a tube coupling the filter component with other components may be housed. In some instances, gear 212(1) may be actuated by a motor, and in turn cause gear 212(2) and/or gear 213(3) to actuate. Accordingly, gear 213(3) may direct filtered fluid into the removal channel. As illustrated in FIG. 2B, gear 213(3) may be coupled to one or more blades, where the blades may direct the filtered fluid into the removal channel. Additionally, or alternatively, below the gear platform 218 with gear system 210 may include a platform 222, which may include filter cavities 220. This way, multiple filters, with collected genetic material attached thereto, may be placed in the filter cavities 220. The platform 222 may also include tube cavity 216(1) and/or tube cavity 216(2), wherein the house cavity 216(1) and/or tube cavity 216(2) may be configured to direct different fluids and/or solutions (e.g., solutions containing dissolved materials from capsules) between the filter component and other components of the fluidic reactor device.

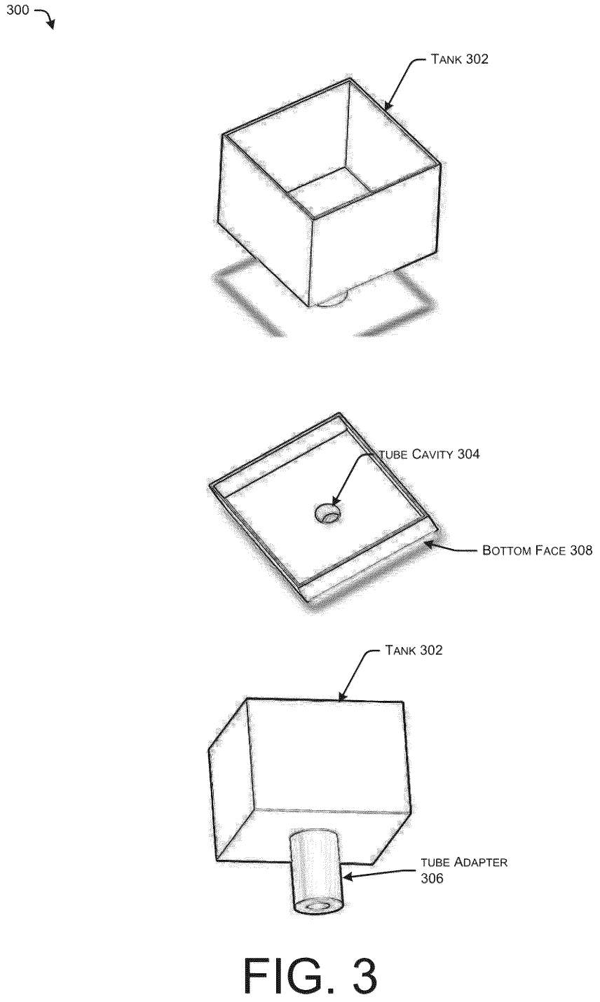

FIG. 3 illustrates an example adapter 300 that may be coupled to the filter component of the fluidic reactor device, according to at least some examples. For example, as illustrated in FIG. 3, the adapter may include a tank 302. The tank 302 may be configured to store collected fluids and/or solvents for further processing of the collected fluids. Additionally, or alternatively, the bottom face 308 of the adapter may include a tube cavity 304 coupled to the tube adapter 306, such that the tank 302 may be coupled with the filter component of the fluidic reactor device. Specifically, the tank 302 may be coupled to the fluid intake component.

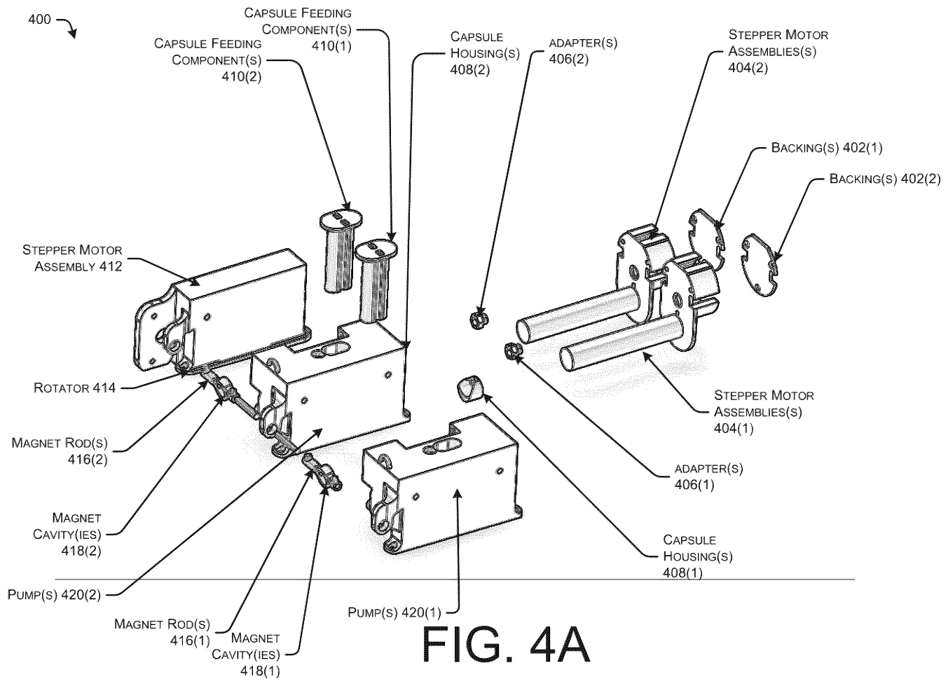

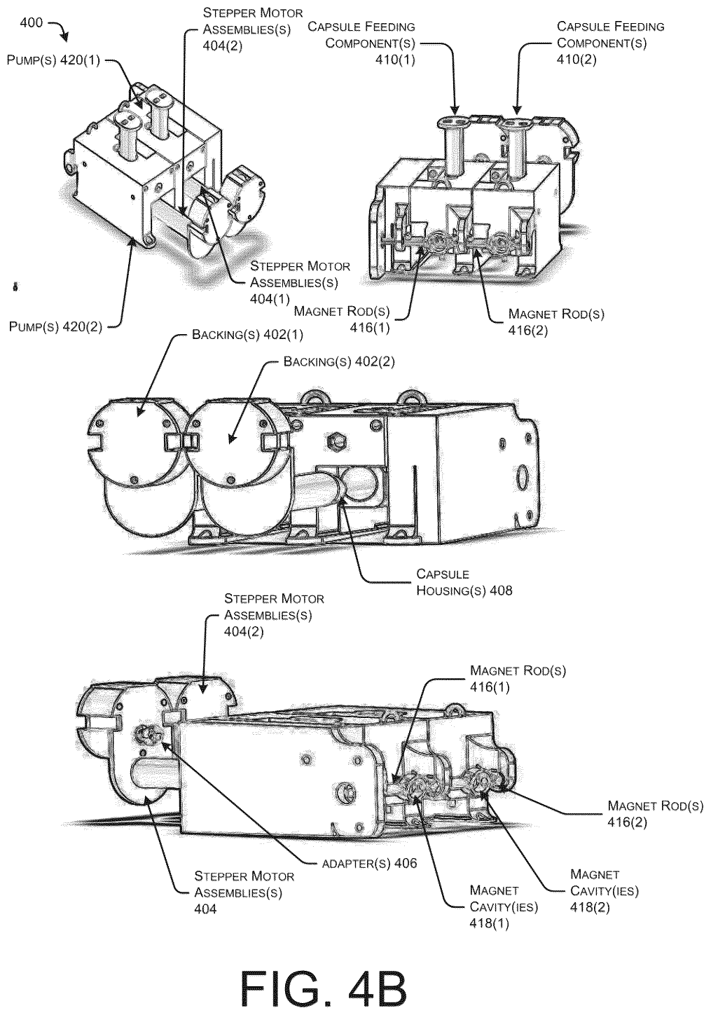

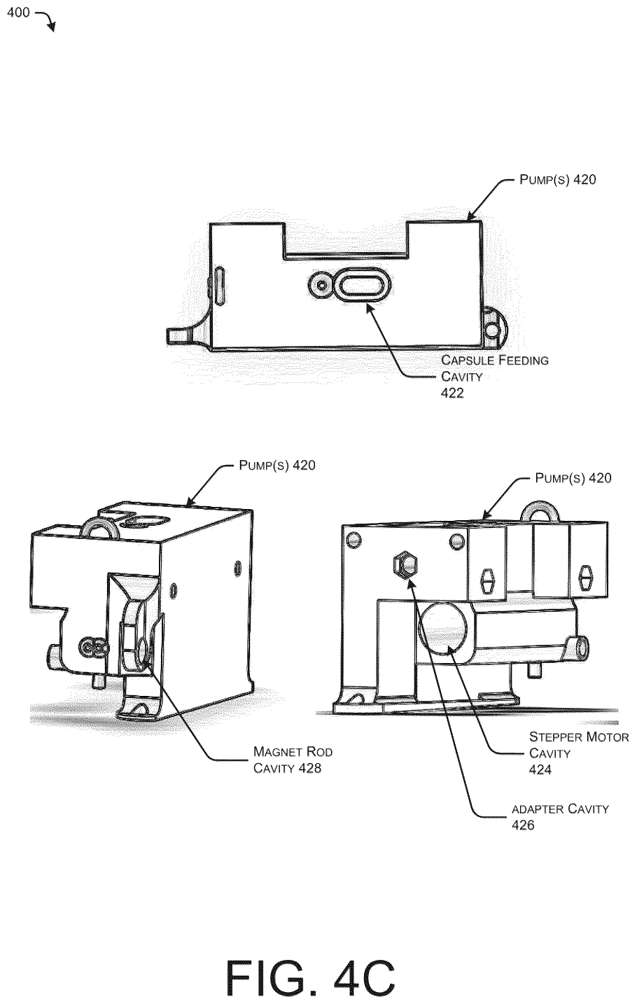

FIGS. 4A-4C illustrate example 400 of modular reactors, or pumps 420, that may be included in the fluidic reactor device, according to at least some examples. In some instances, the filter component, such as filter component 104, may be coupled to one or more pumps, such as pump 420(1) and 420(2). Although not illustrated, the filter component may be coupled to pump 420(1) and/or 420(2) via a tube. Additionally, or alternatively, while FIG. 4A illustrates pumps 420(1) and 420(2), more than two pumps may be utilized and/or implemented. In some examples, pump 420(1) may be configured to generate a solution that is usable, when introduced with a filter, to expel the genetic material from the filter and into the solution. Additionally, or alternatively, pump 420(2) may be configured to generate a solution that may include an enzyme, such as proteinase K, which catalyzes the break-down of proteins (e.g., proteins present in a cell membrane and/or a nuclear membrane). This way, genetic material (e.g., DNA and/or RNA) that is contained within a nucleus may be released into the solution.

In order to generate the cellulase solution at pump 420(1) and/or a proteinase-containing solution at pump 420(2), the pumps 420 may include a capsule feeding component that may store one or more capsules of a lyophilized cellulase, lyophilized proteinase K, and/or another type of enzyme. For example, capsule feeding component 410(1) may store one or more capsules of lyophilized cellulase. Additionally, or alternatively, capsule feeding component 410(2) may store one or more capsules of lyophilized proteinase K. The one or more capsules stored in capsule feeding component 410(1) and/or 410(2) may be configured to dissolve when interacted with fluid and/or solution. Further, the pumps 420 may use, or work in combination with, a motor, such as stepper motor assemblies 404. For example, pump 420(1) may be coupled to stepper motor assembly 404(1), and pump 420(2) may be coupled to stepper motor assembly 404(2). The stepper motor assemblies 404 may include, or house, a stepper motor. The stepper motor assemblies 404 may be coupled to backings, such as backing 402(1) and/or 402(2), where the backings may be configured to secure the stepper motor assemblies 404.

Additionally, or alternatively, the stepper motor assemblies 404 may be coupled to adapters 406. For example, the adapters 406 may be configured to couple the stepper motor included in the stepper motor assemblies 404 to a threaded rod, stock, and/or other linkage. Accordingly, the stepper motor assemblies 404 may be configured to “push” capsules stored in capsule housings 408 into the pumps 420 such that the capsule interacts with a fluid and/or solution inside the pump, such as a fluid and/or solution from the tube, and in turn, cause the capsule to be dissolved and its contents released into the solution. For example, the capsule feeding components 410 may be configured to store and deposit a capsule containing the lyophilized cellulase and/or proteinase K into capsule housings 408. For example, capsule feeding component 410(1) may be configured to deposit a capsule containing lyophilized cellulase into the capsule housing 408(1), and the stepper motor assembly 404(1) may then “push” the capsule into a portion of the pump 420(1) so that the capsule may be dissolved. The dissolved cellulase may then be introduced, such as via a tube, to a filter of the filter components, and cause the filter to degrade and release the genetic material. Specifically, the filter may release cells that include the genetic material. Additionally, or alternatively, capsule feeding component 410(2) may be configured to deposit a capsule containing lyophilized proteinase K into the capsule housing 408(2), and the stepper motor assembly 404(2) may then “push” the capsule into a portion of the pump 420(2) so that the capsule may be dissolved. The dissolved proteinase K may then be introduced, such as via a tube, to the solution containing the genetic material, and cause any cellular membranes to be lysed and genetic material released from any membranes (e.g., the cell membrane and/or nuclear membrane).

Additionally, or alternatively, pump 420(1) and/or 420(2) may use, or work in combination with, an additional motor, such as a stepper motor contained in stepper motor assembly 412. In some examples, pump 420(1) and/or pump 420(2) and the motor may be coupled with a magnet rod, such as magnet rod 416(1) and/or 416(2), which may contain magnet cavities 418 for storing a magnet. Further, inside the pumps 420 may include a magnetic stir bar in proximity to the magnet in the magnet cavities 418, such that the magnet may cause the magnetic stir bar to spin. In some examples, stepper motor assembly 412 may actuate a rotator 414 and thus the spinning of the magnet, and cause the magnetic stir bar to spin. This way, when the capsule interacts with a fluid and/or solution inside the pumps 420, the magnetic stir bar may aid in the dissolution of the capsule. While not illustrated, the pumps 420 may use, or work in combination with, a heating element disposed in, on, or adjacent to the pumps 420 or other reaction chamber to aid in the dissolution of the capsule and/or to subsequently denature an enzyme (e.g., proteinase K). Additionally, or alternatively, as illustrated in FIG. 4C, pump 420(1) and/or 420(2) may contain a capsule feeding cavity 422 that is configured to house the capsule feeding components 410, a magnet rod cavity 428 that is configured to house magnet rods 416, a stepper motor cavity 424 that is configured to house stepper motors in stepper motor assemblies 404, and/or an adapter cavity 426 that is configured to house adapters 406.

In some instances, the pumps 420 may be configured to perform various techniques and reactions in addition to those described above. For example, pump 420(1) may be configured to perform techniques such as loop-mediated isothermal amplification (LAMP). For example, a capsule in the pump 420(1) may contain primers, polymerases, reagents, and/or other materials required for LAMP. As described above, the capsule may be loaded via capsule feeding component 410(1) and pushed into the pump 420(1) by the stepper motor assembly 404(1), and further processed using stirring via rotator 414 and/or heating to dissolve the capsule in a fluid and/or solution. For example, the genetic material, after being released from the filter and further processed using proteinase K, may be introduced to the solution at pump 420(1), where the solution may contain primers, polymerases, reagents, and/or other materials required for LAMP. Subsequently, the genetic material may be amplified using LAMP. Additionally, or alternatively, pump 420(2) may be configured to perform techniques such as the use of Clustered Regularly Interspaced Short Palindromic Repeats (CRISPR) systems with CRISPR-associated protein 12 (Cas12) and/or CRISPR-associated protein 13 (Cas13). For example, as described above, a capsule in the pump 420(2) may contain gRNA, endonucleases, reagents, and/or other materials required for CRISPR-Cas12 and/or CRISPR-Cas13 techniques. The capsule may also include single-stranded genetic material with an associated probe. The capsule may be loaded via capsule feeding component 410(2) and pushed into the pump 420(2) by the stepper motor assembly 404(2), and further processed using stirring via rotator 414 and/or heating to dissolve the capsule in a solution. For example, the genetic material, after being amplified at pump 420(1), may be introduced to the solution at pump 420(2), where the solution may contain gRNA, endonucleases, reagents, single-stranded genetic material with an associated probe, etc. Subsequently, the amplified genetic material may be processed using CRISPR-Cas12 and/or CRISPR-Cas13.

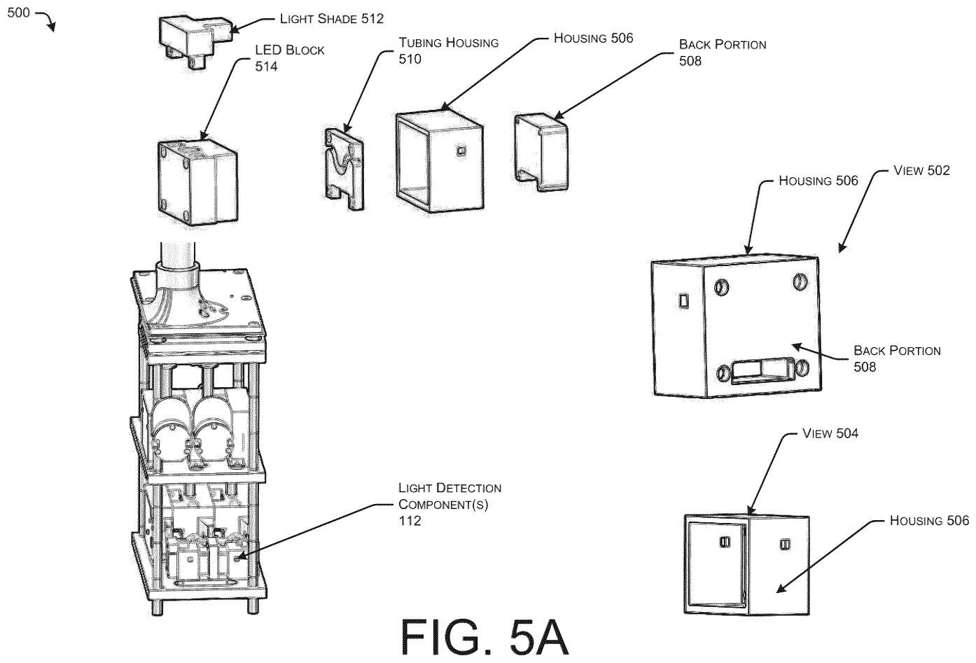

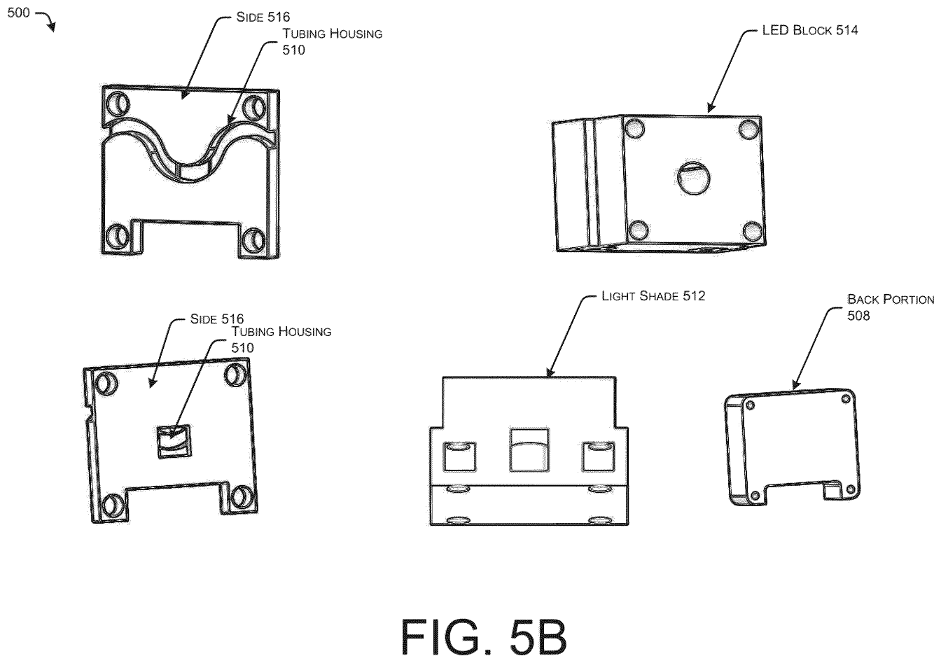

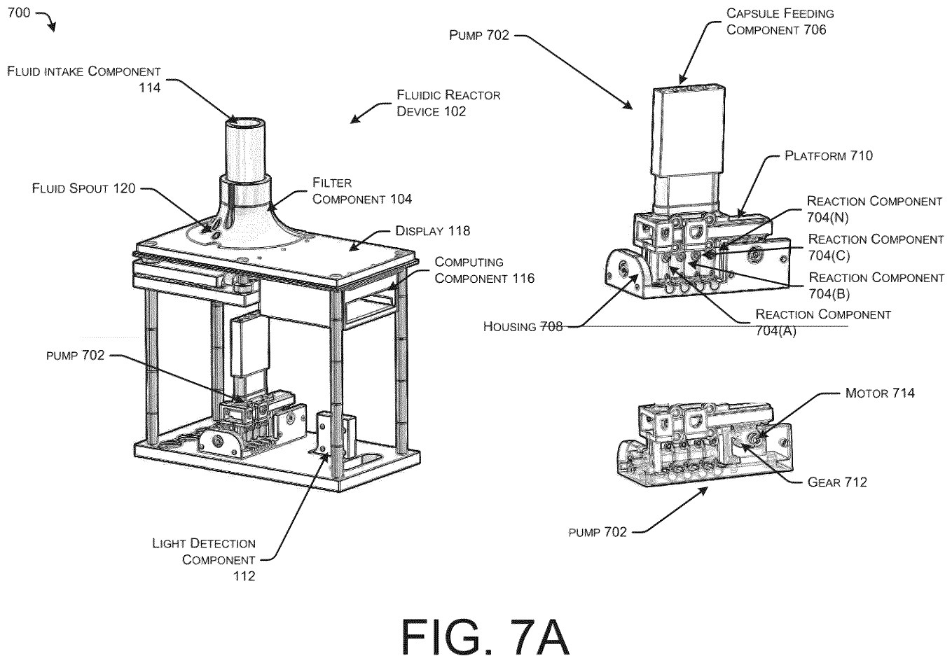

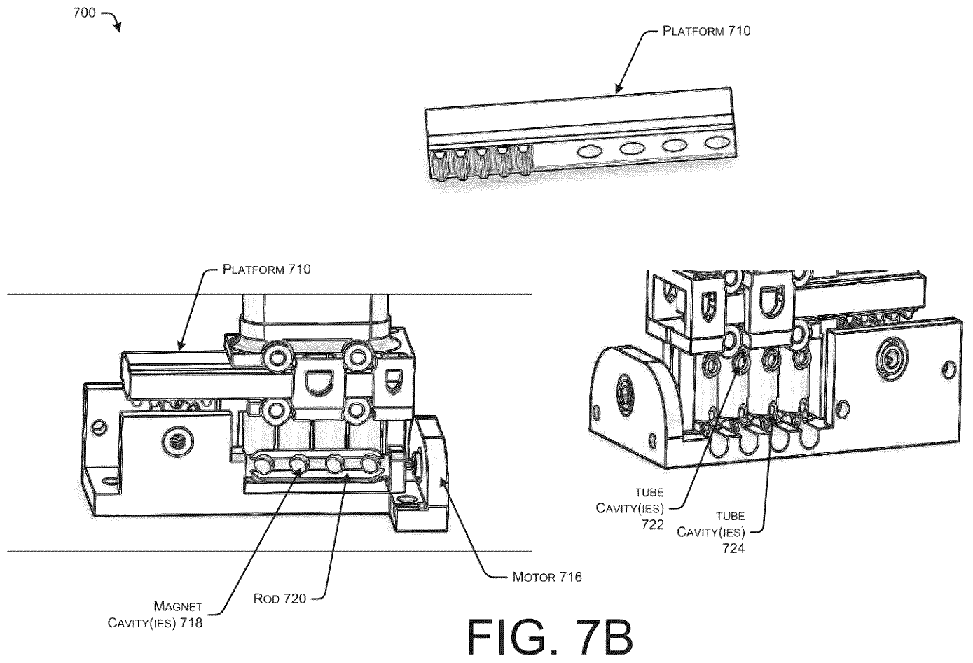

FIGS. 5A and 5B illustrate an example 500 light detection component 112 that may be included in the fluidic reactor device, according to at least some examples. For example, the light detection component may be coupled to the singular pump system, such as the pump system described below in FIGS. 7A and 7B, and/or the multiple pump system as described in FIGS. 4A-4C. While not illustrated, the light detection component 112 may be coupled to the singular pump system and/or the multiple pump system via a tube, where a solution containing the genetic material after CRISPR-Cas12 and/or CRISPR-Cas13 has been performed may be received at the light detection component 112. For example, as described above, in response to the Cas12 and/or Cas13 finding the target sequence and cleaving the genetic material, a voltage change may occur (e.g., when the probe is a charged particle) and/or light may be emitted (e.g., when the probe is a fluorophore). For example, in instances where the probe is a fluorophore, the light detection component 112 may be configured to expose the solution containing the genetic material to light (e.g., blue light and/or light with approximately 400 nanometers wavelength), and cause the emittance of light from the fluorophore (e.g., green light and/or light with approximately 550 nanometers wavelength). For example, the light detection component 112 may include a light-emitting diode (LED) block 514, a light shade 512, tubing housing 510, housing 506, and back portion 508. View 502 illustrates the housing 506 coupled to the back portion 508. View 504 also illustrates the housing 506. In some examples, as illustrated in FIG. 5B, the tubing housing 510 may include side 516 with a groove at which a tube and/or tube containing the genetic material may be contained. Additionally, or alternatively, due to the positioning of the groove in tubing housing 510, at side 516 the tubing housing 510 may include a small window for light to be exposed to the solution containing the genetic material. The tubing housing 510 may then be coupled with LED block 514, light shade 512, and/or back portion 508. Accordingly, a binary result indicating the presence of the target genetic material in the originally collected fluid sample may be produced. In instances where the probe is a fluorophore, if the target genetic material is not present in the originally collected fluid sample, there may be not emittance of light at the light detection component. As such, the binary result may indicate that the target genetic material is not present.

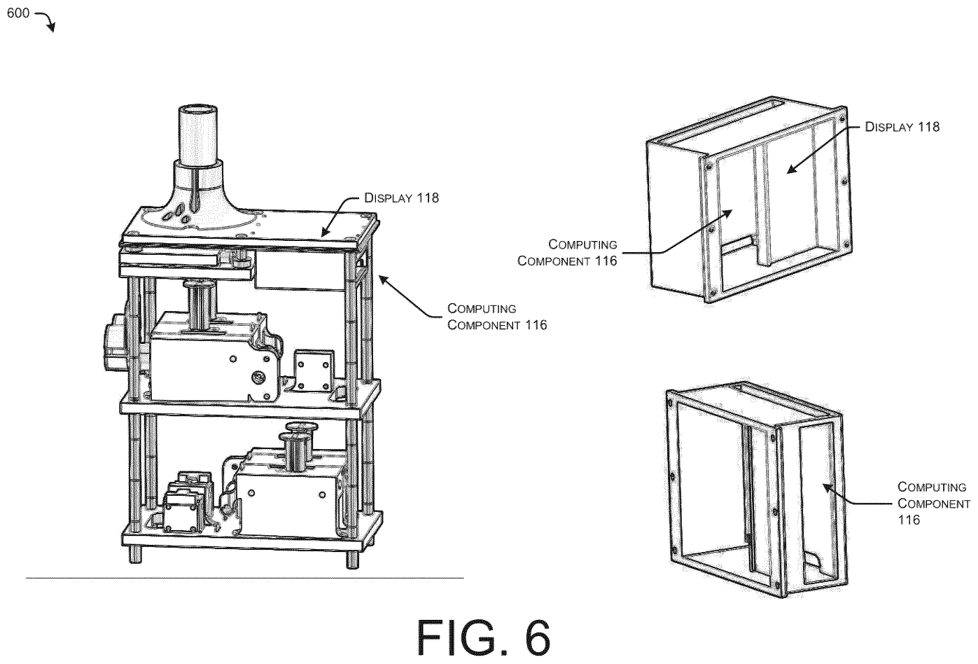

FIG. 6 illustrates an example 600 computing component 116 and display 118 that may be included in the fluidic reactor device, according to at least some examples. For example, the computing component 116 may include processor(s) that may be implemented as appropriate in hardware, computer-executable instructions, firmware, and/or combinations thereof. Computer-executable instructions or firmware implementations of the processor(s) may include computer-executable and/or machine-executable instructions written in any suitable programming language to perform the various functions and/or techniques described. The computing component 116 may also include memory configured to store program instructions that are loadable and executable on the processor(s), as well as data generated during the execution of these programs. The computing component 116 may also include storage, such as either removable storage or non-removable storage including, but not limited to, magnetic storage, optical disks, and/or tape storage. The computing component 116 may be coupled to a remote computing device (e.g., mobile device, laptop, computer, etc.) via network interfaces and over a local network, internet connection, BLUETOOTH®, and/or or other wireless connectivity. The computing component 116 may be capable of establishing a communication session with a remote computing device and/or transmitting/receiving data from that remote computing device.

The display 118 may include a communications interface that may include physical buttons (e.g., mechanical, resistive, capacitive, etc.). touch screens, etc. The communications interface of the display 118 may also include communications with the computing component 116 and/or the remote computing device to provide instructions to the fluidic reactor device. Additionally, or alternatively, the remote computing device may be configured to provide instructions to the fluidic reactor device via the computing component 116 and/or the communications interface of the display 118.

FIGS. 7A and 7B illustrate another example 700 modular reactor, or pump 702, that may be included in the fluidic reactor device 102, according to at least some examples. It is to be appreciated that the same components and/or techniques described above with respect to FIGS. 4A-4C may be similarly used and/or performed using the singular pump 702. In some examples, the pump 702 may contain one or more reaction components 704 configured to receive one or more capsules, such as the capsules described above, and perform various reactions therein. For example, the pump 702 may include reaction component 704(A), 704(B) 704(C), and/or 704(N) (where “N” is any integer greater than one or letter beyond “A”). For example, reaction component 704(A) may be configured to receive a capsule from capsule feeding component 706 containing lyophilized enzyme and produce a solution with the enzyme configured to catalyze the decomposition of the filter at the filter component 104. For example, in instances where the filter is a cellulose filter, reaction component 704(A) may be configured to receive a capsule from the capsule feeding component 706 containing lyophilized cellulase enzyme, such that the cellulase solution, as described above, may be produced in the reaction component 704(A) and configured to catalyze the decomposition of the cellulose filter at the filter component 104. In another example, reaction component 704(B) may be configured to receive a capsule containing lyophilized proteinase K from the capsule feeding component 706, such that the proteinase-containing solution, as described above, may be produced in the reaction component and configured to lyse cellular membranes containing the genetic material. In another example, reaction component 704(C) may be configured to receive a capsule containing the materials and/or reagents required for LAMP techniques from the capsule feeding component 706, such that the genetic material, as described above, may be amplified. In another example, reaction component 704(N) may be configured to receive a capsule containing the materials and/or reagents required for CRISPR-Cas12 and/or CRISPR-Cas13 techniques from the capsule feeding component 706, such that target genetic material, as described above, may be detected. The one or more reaction components may be coupled to the filter component and/or other components via a tube. For example, tube cavity 722 may house a tube such that a fluid and/or solution may be pumped into the reaction chamber, such that the capsules may be dissolved and/or reactions may take place. In another example, tube cavity 724 may house a tube such that the fluid and/or solution after the capsules are dissolved and/or reactions taken place may be pumped to other components, such as the filter component 104, the light detection component 112, and/or reaction components 704.

While the singular pump 702 may use any of the components, techniques, and/or mechanisms described above with the multiple-pump system, the singular pump may include, but is not limited to, a capsule feeding component 706, platform 710, housing 708, motor(s) 714, and/or gear(s) 712 to perform the techniques described herein. For example, the capsule feeding component 706 may store one or more capsules containing the materials and/or reagents as described above. Further, the pump 702 may use, or work in combination with, a motor 714, such as a stepper motor. In some examples, the motor 714 may be coupled to a capsule housing 708 containing the one or more reaction components 704 and a gear 712. The motor 714 and gear 712 may also be coupled to a platform 710, where the platform 710 may be disposed between the capsule feeding component 706 and the housing 708. Accordingly, the motor 714 and gear 712 may be configured to actuate and/or otherwise move the platform 710 such that the one or more capsules may be deposited into the one or more reaction components 704, and techniques described above may be performed. Additionally, or alternatively, as illustrated in FIG. 7B, the pump 702 may similarly include magnet cavities 718, rod 720, and motor 716. For example, motor 716 may actuate a rotator and thus cause the rod 720 to spin, as well as the magnets disposed in the magnet cavities 718. This way, when the capsule interacts with a fluid and/or solution inside the pump reaction components 704, the magnetic stir bar may aid in the dissolution of the capsule and/or other reactions.

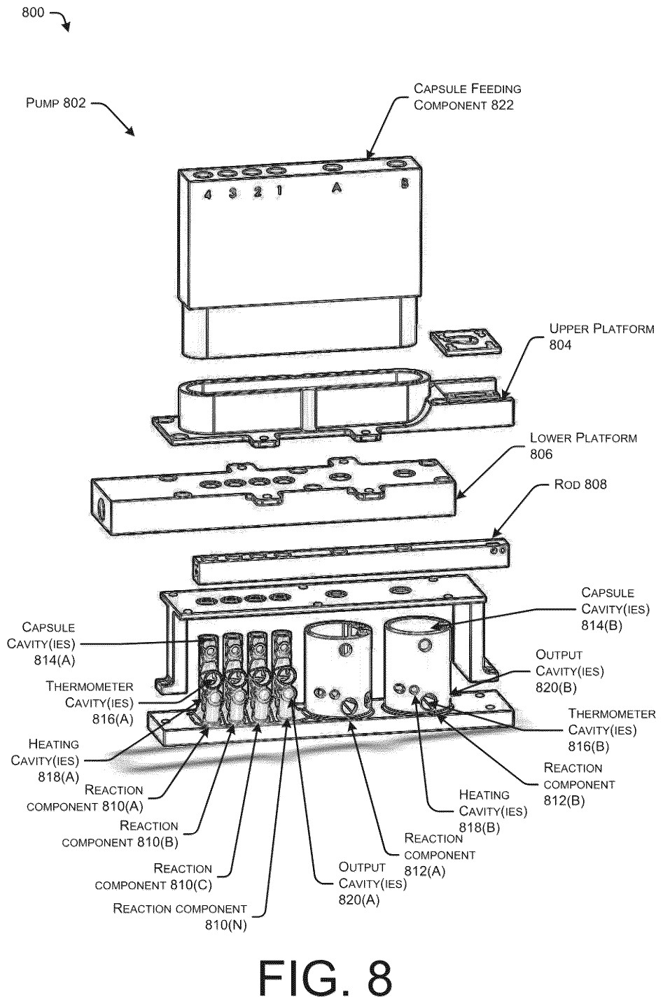

FIG. 8 illustrates another example 800 module reactor, or pump 802, which may be included in the fluidic reactor device 102, according to at least some examples. It is to be appreciated that the same components and/or techniques described above with respect to FIGS. 4A-4C, 7A, and 7B may be similarly used and/or performed using the pump 802. In some examples the pump 802 may contain one or more reaction components (e.g., reaction components 810 and reaction components 812), configured to receive one or more capsules, such as the capsules described above, and perform various reactions therein. For example, the pump 802 may include reaction component 810(A), 810(B), 810(C), and/or 810(N) (where “N” is any integer greater than one or letter beyond “A”). By way of example, and not limitation, reaction component 810(A) may be configured to receive a capsule from capsule feeding component 822, where the capsule may include an isothermal solution (e.g., materials and/or reagents required for LAMP techniques). The capsule feeding component 822 may use, or work in combination with, upper platform 804, lower platform 806, and/or rod 808 to cause one or more capsules to be deposited into reaction component 810(A). For example, the capsule feeding component 822 may store one or more capsules containing materials and/or reagents as described above. While not illustrated in FIG. 8, the capsule feeding component 822 may use, or work in combination with, a motor (e.g., linear actuator) that is coupled to the upper platform 804, lower platform 806, and rod 808. Accordingly, a motor may be configured to actuate and/or other move the rod 808 such that the one or more capsules may be deposited into the reaction components 810, such as reaction component 810(A). Additionally, or alternatively, reaction components 810 may include capsule cavity(ies) 814(A), thermometer cavity(ies) 816(A), and/or heating cavity(ies) 818(A). By way of example, and not limitation, capsules from capsule feeding component 822 may be deposited into reaction components 810 using the capsule cavity(ies) 814(A). As described above with respect to FIGS. 7A and 7B, upon receipt of a capsule containing materials and/or reagents (e.g., lyophilized cellulase enzyme, lyophilized proteinase K, reaction component 810(A), materials and/or agents required for LAMP techniques, materials and/or reagents required for CRISPR-Cas12 and/or CRISPR-Cas13 techniques, and/or the like) subsequent reactions may be performed in the reaction components 810. In some instances, while not illustrated in FIG. 8, a thermometer may be coupled to the reaction components 810 by way of the thermometer cavity(ies) 816(A) and configured to monitor the temperature of a reaction taking place in reaction components 810 using one or more capsules from capsule feeding component 822. Additionally, or alternatively, while not illustrated in FIG. 8, a heating element may be coupled to the reaction components 810 by way of the heating cavity(ies) 818(A) and configured to aid in the dissolution of the capsule and/or to subsequently denature an enzyme (e.g., proteinase K). Reaction components 810 may also include output cavity(ies) 820(A) that are configured to move solutions from the reaction components 810 (e.g., from reaction component 810(A) to reaction component 810(B), from reaction component 810(C) to another component of the fluidic reactor device 102, etc.). For example, output cavity(ies) 820(A) may be coupled to one or more hoses between components of the fluidic reactor device 102. Additionally, or alternatively, reaction components 812, capsule cavity 814(B), thermometer cavity(ies) 816(B), heating cavity(ies) 818(B), and/or output cavity(ies) 820(B) may similar be used as reaction components 810, thermometer cavity(ies) 816(A), heating cavity(ies) 818(A), and/or output cavity(ies) 820(A).

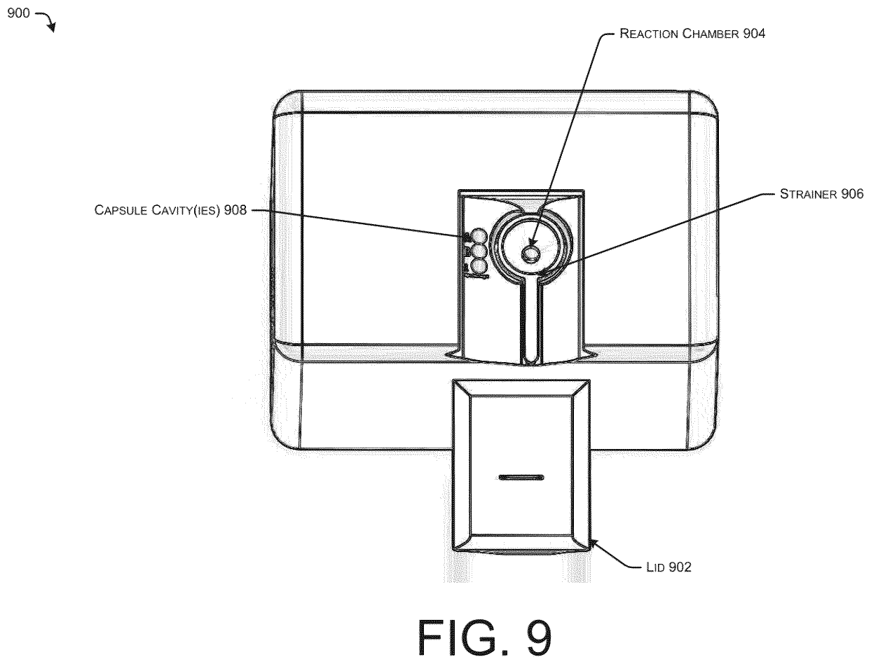

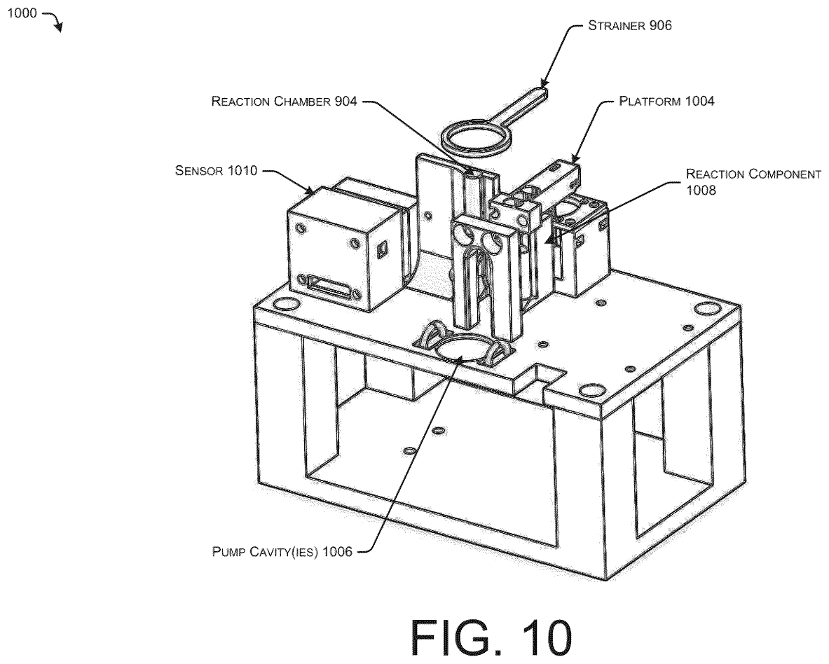

FIG. 9 illustrates an example sampling component 900 that may be included in, or work in combination with, the fluidic reactor device 102, according to at least some examples. In some instances, the sampling component 900 may be used in combination with the fluidic reactor device 102 to collect and/or receive fluid from the environment and/or other samples for processing. For example, sampling component 900 may include lid 902, reaction chamber 904, strainer 906, and/or capsule cavity(ies) 908. The lid 902 may be configured to protect and/or cover the chamber 904, strainer 906, and/or capsule cavity(ies) 908. The chamber 904 (e.g., a reaction component) may be configured to receive a fluid and/or other sample, where the fluid and/or other sample may first pass through the filter 906. For example, a sample (e.g., cotton swab, biological material, etc.) may be placed into the strainer along with a fluid (e.g., water) so as to dispel genetic material (e.g., eDNA, RNA, etc.) into a solution and the reaction chamber 904 for further processing according to the techniques described herein and as described in more detail below with respect to FIG. 10. Additionally, or alternatively, the sampling component may receive one or more capsules via capsule cavity(ies) 908.

FIG. 10 illustrates an example 1000 module reactor (e.g., reaction component 1008) and light detection component (e.g., sensor 1010) that may be included in the fluidic reactor device 102 and used in combination with the sampling component 900 of FIG. 9, according to at least some examples. For example, and as described above with respect to FIG. 9, the sampling component 900 may include a strainer 906 that is coupled to reaction chamber 904. Additionally, or alternatively, the reaction chamber 904 may include, or be coupled to, platform 1004 and/or reaction component 1008. While not illustrated, in some instances, a motor (e.g., stepper motor) may be configured to actuate and/or otherwise move the platform 1004 such that one or more capsules (e.g., containing materials and/or reagents), genetic material in solution, etc. from the sampling component 900 may be deposited into one or more reaction components, such as reaction component 1008, such that one or more reactions may be performed according to the techniques described herein. By way of example, and not limitation, the sampling component 900 may receive a sample (e.g., cotton swab) in combination with a fluid (e.g., water), where the sample and fluid may pass through the filter 906 and into the reaction chamber 904 that may be coupled to reaction component 1008. The performance of a reaction (e.g., LAMP techniques, CRISPR-Cas12 techniques, CRISPR-Cas13 techniques, etc.) such that target genetic material may be detected may occur in the reaction chamber 904.

Additionally, or alternatively, a light detection component, such as sensor 1010, may be coupled to the reaction chamber 904 and/or reaction component 1008, either as a singular pump system or multiple pump system. The sensor 1010 may be coupled to the reaction chamber 904 and/or reaction component 1008 via a tube, where a solution containing genetic material after CRISPR-Cas12 and/or CRISPR-Cas13 has been performed may be received by the sensor 1010. The sensor 1010 may comprise a voltage detection component, where in response to the Cas12 and/or Cas13 finding the target sequence and cleaving the genetic material, a voltage change may occur (e.g., when the probe is a charged particle) and/or light may be emitted (e.g., when the probe is a fluorophore). For example, in instances where the probe is a fluorophore, the sensor 1010 may be configured to expose the solution containing the genetic material to light (e.g., blue light and/or light with approximately 400 nanometers wavelength), and cause the emittance of light from the fluorophore. Accordingly, a binary result indicating the presence of target genetic material may be output by the sensor 1010. Additionally, or alternatively, pump cavity(ies) 1006 may be configured to couple the reaction chamber 904, reaction component 1008, sensor 1010, and/or other components of the fluidic reactor device 102 to one or more pumps such that fluid may move throughout various components of the fluidic reactor device 102.

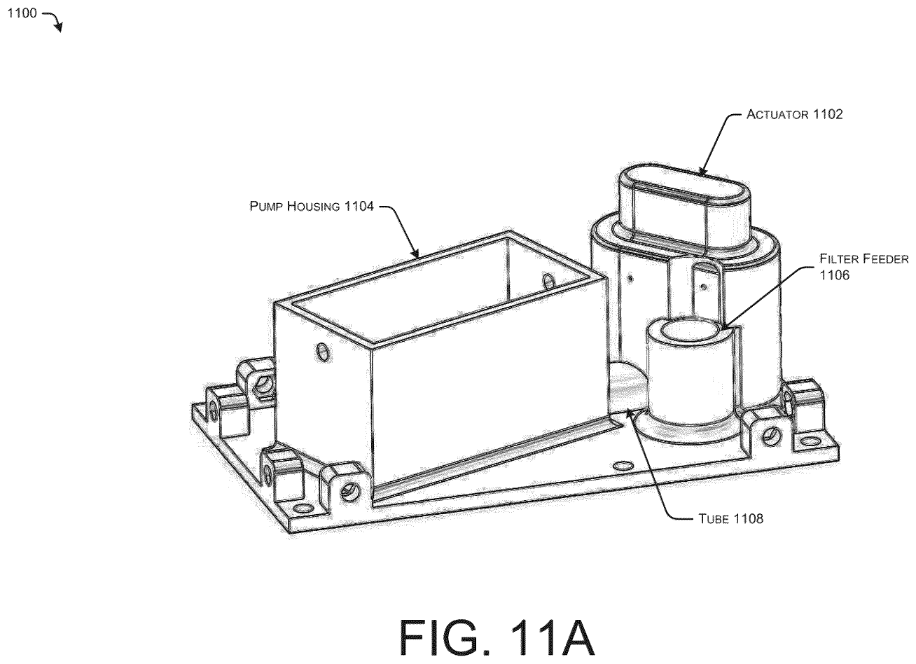

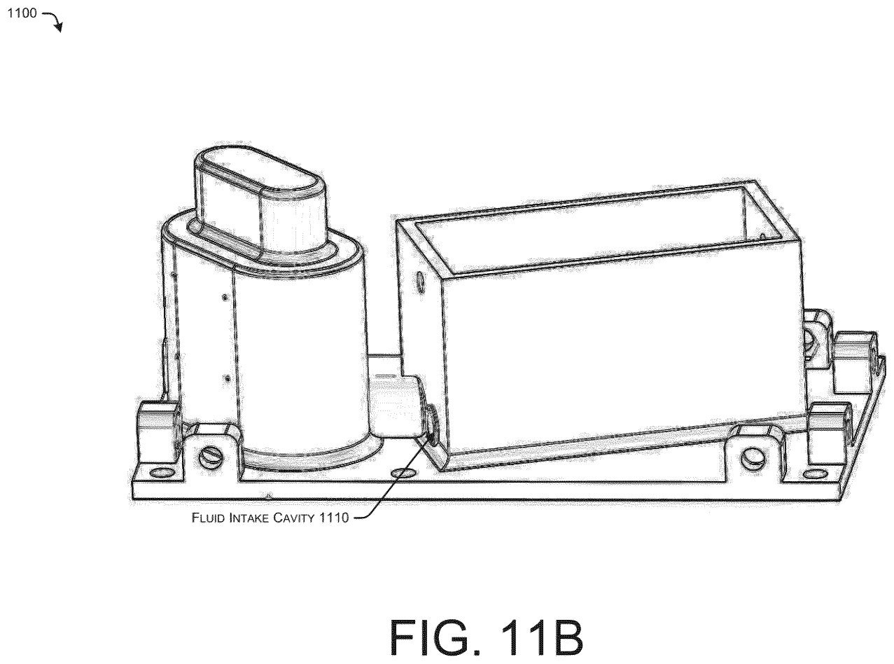

FIGS. 11A and 11B illustrate additional examples 1100 of components to be included in the fluidic reactor device 102, according to at least some examples. For example, the fluidic reactor device may include an actuator 1102, water pump housing 1104, filter feeder 1106, tube 1108, and/or fluid intake cavity 1110. In some instances, the water pump housing 1104 may be configured to house a fluid intake component (e.g., fluid intake component 114) which may collect a fluid from the environment using fluid intake cavity 1110 (e.g., a tube connected to the water pump housing 1104 via fluid intake cavity 1110). Fluid may be collected from the environment via the fluid intake cavity 1110 by a fluid intake component of the water pump housing 1104 working in combination with a siphon, pump, vacuum, fan, funnel, etc. The water pump housing 1104 may also be coupled to an actuator 1102 via tube 1108. In some instances, the actuator may include a linear actuator (e.g. solenoids). In some instances, the actuator 1102 may be configured to actuate the placement of filters in a filter component via the filter feeder 1106. The filter feeder 1106 may be configured to store one or more filters for use in a filter component (e.g., filter component 104). For example, once the fluid has been collected and disposed in a filter component, the fluid may subsequently be filtered through a filter. In some examples, the filter may comprise cellulose, though in other examples filters may additionally or alternatively include one or more other materials (e.g., polymers, ceramics, metals, etc.). The filter may be placed in the filter component via the filter feeder 1106, where the filter feeder 1106 may store one or more filters.

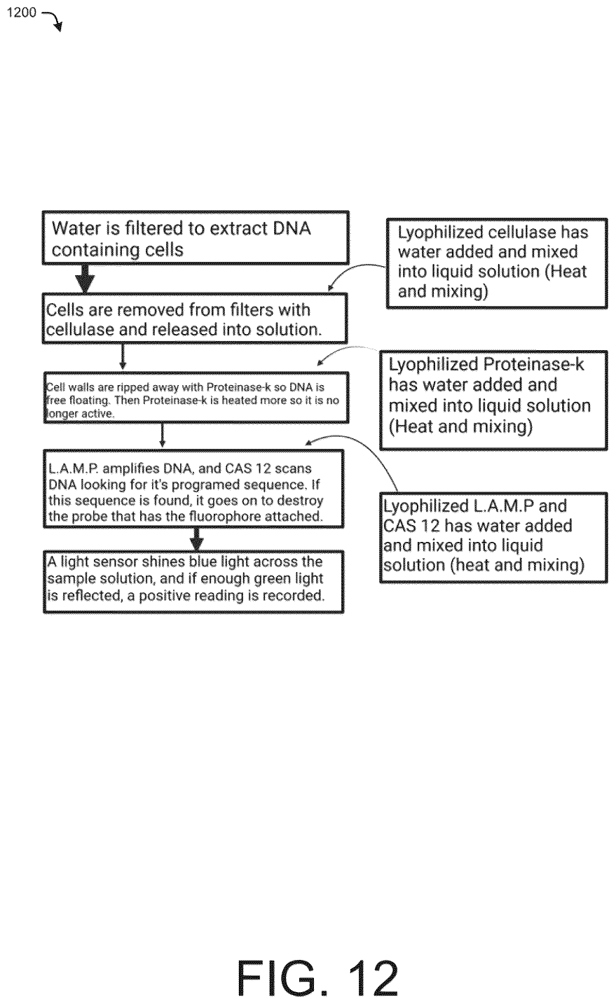

FIG. 12 illustrates a flow diagram of an example process 1200 for a fluidic reactor device to detect genetic material in a sample, according to at least some examples. For example, water may be filtered to extract DNA containing cells. Additionally, or alternatively, lyophilized cellulase may be combined with water and mixed into a liquid solution (e.g., via heating and mixing via a magnetic stir bar). The cells may be removed from the filters via the cellulase, and released into a solution. Additionally, or alternatively, lyophilized proteinase K may be combined with water and mixed into a liquid solution (e.g., via heating and mixing via a magnetic stir bar). This way, cell walls and/or cellular membranes may be ripped away with proteinase K so that the DNA is “free floating.” Subsequently, the proteinase K may be heated more so as to be denatured. Lyophilized LAMP reagents and Cas12 may be combined with water and mixed into a liquid solution (e.g., via heating and mixing via a magnetic stir bar). LAMP may then be performed to amplify DNA, and Cas12 and/or Cas13 may scan the DNA looking for its programmed sequence. If the sequence is found, then a probe, such as a fluorophore, may emit light due to its associated single-stranded genetic material being destroyed. A light sensor may then shine a blue light across the sample solution, and if enough green light is reflected, a positive reading is recorded.

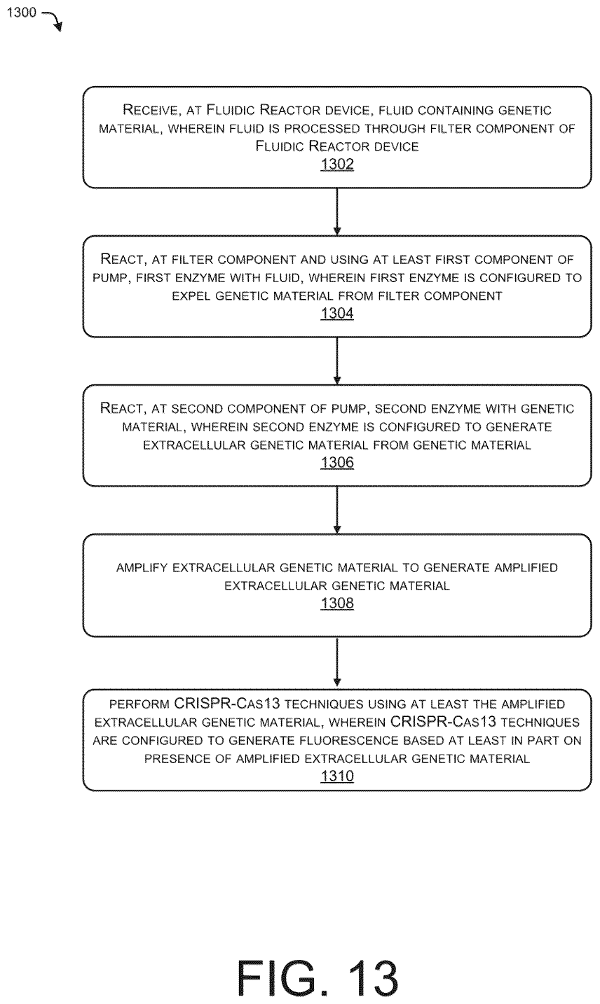

FIG. 13 illustrates a flow diagram of another example process 1300 for a fluidic reactor device to detect genetic material in a sample, according to at least some examples. The techniques may be applied by a system comprising one or more processors, and one or more non-transitory computer-readable media storing computer-executable instructions that, when executed by the one or more processors, cause the one or more processors to perform the operations of process 1300.

The processes described herein are illustrated as collections of blocks in logical flow diagrams, which represent a sequence of operations, some or all of which may be implemented as hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures and systems described in the examples herein, although the processes may be implemented in a wide variety of other environments, architectures and systems.

At block 1302, the process 1300 may include receiving, at the fluidic reactor device, a fluid containing genetic material, wherein the fluid is processed through a filter component of the fluidic reactor device. By way of example, and not limitation, the fluidic reactor device may be disposed in an environment, such as in or near a waterway, wastewater system, ventilation system, open air environment, and/or any other environmental feature where genetic material may be detected in a fluid (e.g., gas, liquid, etc.). In some examples, the fluid intake component may include, or work in combination with, a siphon, pump, vacuum, fan, funnel, etc. to collect a fluid from the environment, where the fluid intake component may cause the fluid to be collected and disposed in the filter component of the fluidic reactor device. The fluid intake component or portion thereof may be actively driven (e.g., by a motor) to pull fluid into the reactor, or may passively collect fluid using the flow of the fluid or other naturally occurring forces or conditions (flow of a waterway, wastewater system, siphon, flow of air through a ventilation duct, wind, etc.). Additionally, or alternatively, the fluid intake component may be associated with one or more adapters configured to aid in the collection of a fluid from the environment. For example, an adapter may be coupled to the fluid intake component such that the fluidic reactor device may be adapted to different scenarios, environments, etc. For example, the adapter may include a tank that is configured to store collected fluids and/or solvents for further processing of the collected fluids before an interaction with the filter component of the fluidic reactor device. In some examples, the fluid intake component may collect fluid continuously or periodically.

Once the fluid has been collected and disposed in the filter component, the fluid may subsequently be filtered through a filter. In some examples, the filter may comprise cellulose, though in other examples filters may additionally or alternatively include one or more other materials (e.g., polymers, ceramics, metals, etc.). The filter may be placed in the filter component via a filter feeder, where the filter feeder may store one or more filters to be loaded into the filter component. The filter may be configured to collect and/or attract genetic material (e.g., cells containing DNA and/or RNA) that is present in the fluid. For example, the filter may collect and/or attract the genetic material due to hydrogen bonding, hydrophobic interactions, and/or other types of bonding and/or intermolecular forces. Additionally, or alternatively, the filter component may include a spout for filtered fluid. In some examples, the spout may be configured to discharge the fluid once the fluid has been filtered through the filter of the filter component. Additionally, or alternatively, the filter component may include, or work in combination with, one or more gears, wherein the one or more gears are configured to direct filtered fluid into a removal channel, where the fluid is then discharged via the spout. For example, one of the gears may be actuated by a motor, such as a stepper motor. In some examples, another gear may be coupled to one or more blades, where the blades may direct the filtered fluid into the removal channel. Additionally, or alternatively, the filter component may include a platform with one or more filter cavities, such that multiple filters, with collected genetic material attached thereto, may be placed in the filter cavities. This way, one or more filters may be rotated and/or shifted for further processing.

At block 1304, the process 1300 may include reacting, at the filter component and using at least a first component of a pump, a first enzyme with the fluid, wherein the first enzyme is configured to expel the genetic material from the filter component. In some instances, the filter component may be coupled to one or more pumps (or “reactors”). For example, the filter component may be coupled to the one or more pumps via a tube. The pumps may comprise pressure-driven pumps. In some instances, one pump may be configured to generate a solution that is usable, when introduced with a filter, to expel the genetic material from the filter and into the solution. In some instances, the genetic material may be expelled from the filter and into the solution at the filter component. For example, when a cellulose filter is used, the solution may include an enzyme, such as cellulase, that catalyzes decomposition of the cellulose filter, and in turn, causes the genetic material collected by the cellulose filter (e.g., cells containing DNA and/or RNA) to be released into the solution. In the case of other types of filter materials, other types of enzymes or techniques may be used to release the genetic material from the filter media.

In order to generate the cellulase solution and/or a proteinase-containing solution (and/or other enzyme-containing solutions for different filter types), the pump may include a capsule feeding component that may store one or more capsules of a lyophilized cellulase, lyophilized proteinase K, and/or another type of enzyme. In some examples, the one or more capsules may be configured to dissolve when interacted with fluid and/or solution. Further, the pump may use, or work in combination with, a motor, such as a stepper motor. In some examples, the capsule feeding component may be configured to deposit a capsule containing the lyophilized cellulase and/or proteinase K into a capsule housing that is coupled with the motor. Additionally, or alternatively, the pump and the motor may be coupled with a threaded rod, stock, and/or other linkage. This way, the motor may be configured to “push” the capsule into the pump such that the capsule interacts with a solution inside the pump, such as a fluid and/or solution from the tube, and in turn, causes the capsule to dissolve. Additionally, or alternatively, the pump may use, or work in combination with, an additional motor, such as a stepper motor. In some examples, the pump and the motor may be coupled with a magnet rod, which may contain one or more cavities for storing a magnet. Further, inside the pump may include a magnetic stir bar in proximity to the magnet, such that the magnet may cause the magnetic stir bar to spin. In some examples, the motor may actuate the spinning of the magnet, and thus cause the magnetic stir bar to spin. This way, when the capsule interacts with a solution inside the pump, the magnetic stir bar may aid in the dissolution of the capsule. Additionally, or alternatively, the pump may use, or work in combination with, a heating element disposed in, on, or adjacent to the pump or other reaction chamber to aid in the dissolution of the capsule and/or to subsequently denature an enzyme (e.g., proteinase K).

In one non-limiting example, a capsule containing a lyophilized cellulase enzyme may be dissolved in a solution at a first pump using the components and/or techniques described above. The cellulase solution may then be introduced, via a tube, to the filter component such that a cellulase filter containing genetic material may decomposed and/or degraded, and cause genetic material to be released in solution.

At block 1306, the process 1300 may include reacting, at a second component of the pump, a second enzyme with the genetic material, wherein the second enzyme is configured to generate extracellular genetic material from the genetic material. For example, a solution may include an enzyme, such as proteinase K, which catalyzes the break-down of proteins (e.g., proteins present in a cell membrane and/or a nuclear membrane) via the hydrolysis of peptide bonds. This way, genetic material that is contained within a nucleus of a cell may be released into the fluid and/or solution as extracellular genetic material.

In order to generate the cellulase solution and/or a proteinase-containing solution (and/or other enzyme-containing solutions for different filter types), the pump may include a capsule feeding component that may store one or more capsules of a lyophilized cellulase, lyophilized proteinase K, and/or another type of enzyme. In some examples, the one or more capsules may be configured to dissolve when interacted with fluid and/or solution. Further, the pump may use, or work in combination with, a motor, such as a stepper motor. In some examples, the capsule feeding component may be configured to deposit a capsule containing the lyophilized cellulase and/or proteinase K into a capsule housing that is coupled with the motor. Additionally, or alternatively, the pump and the motor may be coupled with a threaded rod, stock, and/or other linkage. This way, the motor may be configured to “push” the capsule into the pump such that the capsule interacts with a solution inside the pump, such as a fluid and/or solution from the tube, and in turn, causes the capsule to dissolve. Additionally, or alternatively, the pump may use, or work in combination with, an additional motor, such as a stepper motor. In some examples, the pump and the motor may be coupled with a magnet rod, which may contain one or more cavities for storing a magnet. Further, inside the pump may include a magnetic stir bar in proximity to the magnet, such that the magnet may cause the magnetic stir bar to spin. In some examples, the motor may actuate the spinning of the magnet, and thus cause the magnetic stir bar to spin. This way, when the capsule interacts with a solution inside the pump, the magnetic stir bar may aid in the dissolution of the capsule. Additionally, or alternatively, the pump may use, or work in combination with, a heating element disposed in, on, or adjacent to the pump or other reaction chamber to aid in the dissolution of the capsule and/or to subsequently denature an enzyme (e.g., proteinase K).

Additionally, or alternatively, a capsule containing a lyophilized proteinase K enzyme may be dissolved in a solution at a second pump using the components and/or techniques described above. The proteinase K solution may then be introduced, via a tube, to the released genetic material in solution such that any membranes containing the genetic material may be broken-down, or lysed, and the remaining proteinase K enzyme is denatured via the application of heat. This way, the genetic material may be extracellular.

At block 1308, the process 1300 may include amplifying the extracellular genetic material to generate amplified extracellular genetic material. For example, in some instances, one additional pump may be configured to perform techniques such as loop-mediated isothermal amplification (LAMP). For example, a capsule in a first additional pump may contain primers, polymerases, reagents, and/or other materials required for LAMP. As described above, the capsule may be loaded, or “pushed” into the first additional pump, and further processed using stirring and/or heating to dissolve the capsule in solution. Additionally, or alternatively, the genetic material, after being released from the filter and further processed using proteinase K, may be introduced to the solution at the first additional pump, where the solution may contain primers, polymerases, reagents, and/or other materials required for LAMP. Subsequently, the genetic material may be amplified using LAMP.

At block 1310, the process 1300 may include performing CRISPR-Cas13 techniques using at least the amplified extracellular genetic material, wherein the CRISPR-Cas13 techniques are configured to generate fluorescence based at least in part on a presence of the amplified extracellular genetic material. For example, a second additional pump may be configured to perform techniques such as the use of Clustered Regularly Interspaced Short Palindromic Repeats (CRISPR) systems with CRISPR-associated protein 12 (Cas12) and/or CRISPR-associated protein 13 (Cas13). For example, a capsule in the second additional pump may contain the targeted genetic material. For example, the capsule may contain guide RNA (gRNA) including a nucleotide spacer sequence indicative of the targeted genetic material (e.g., DNA and/or RNA). The capsule may also contain endonucleases (e.g., Cas12 and/or Cas13), reagents, and/or other materials required for CRISPR-Cas12 and/or CRISPR-Cas13 techniques. Additionally, or alternatively, the capsule may contain single-stranded genetic material (e.g., ssDNA and/or ssRNA). The single-stranded genetic material may contain, or be attached with, a probe, such as a charged particle and/or fluorophore. As described above, the capsule may be loaded, or “pushed” into the second additional pump, and further processed using stirring and/or heating to dissolve the capsule in solution. Additionally, or alternatively, the amplified genetic material may be introduced to the solution at the second additional pump, where the amplified genetic material may be further processed using CRISPR-Cas12 and/or CRISPR-Cas13.

By way of example, and not limitation, if the amplified genetic material contains the sequence indicated by the gRNA, the Cas12 and/or Cas13 may be configured to cleave the genetic material (e.g., cleave the strands of DNA). Additionally, or alternatively, all local single-stranded genetic material present may be destroyed, such as the single-stranded genetic material attached with a probe. Accordingly, in response to the Cas12 and/or Cas13 finding the target sequence and cleaving the genetic material, a voltage change may occur (e.g., when the probe is a charged particle) and/or light may be emitted (e.g., when the probe is a fluorophore) due to the probe being cleaved from the single-stranded genetic material. In another example, if the amplified genetic material does not contain the sequence indicated by the gRNA, genetic material may not be cleaved and/or otherwise destroyed, and as such, no voltage change and/or light emittance may occur.

Additionally, or alternatively, the process 1300 may further include wherein the first enzyme is a cellulase enzyme and the second enzyme is a proteinase enzyme.

Additionally, or alternatively, the process 1300 may further include wherein the amplifying comprises loop-mediated isothermal amplification (LAMP).

Additionally, or alternatively, the process 1300 may further include detecting, at a light detection component associated with the fluidic reactor device, fluorescence indicating the presence of the amplified extracellular genetic material, and receiving, at a computing system associated with the fluidic reactor device, data indicating the presence of the amplified extracellular genetic material based at least in part on the fluorescence meeting or exceeding a threshold.

Additionally, or alternatively, the process 1300 may further include detecting, at a light detection component associated with the fluidic reactor device, a lack of the fluorescence indicating the presence of the amplified extracellular genetic material, and receiving, at a computing system associated with the fluidic reactor device, data indicating a lack of the presence of the amplified extracellular genetic material based at least in part on the fluorescence being below a threshold.

Additionally, or alternatively, the process 1300 may further include wherein reacting the first enzyme with the fluid and reacting the second enzyme with the genetic material comprises an application of heat and/or mixing reagents.

Additionally, or alternatively, the process 1300 may further include wherein the fluid is first fluid, the genetic material is first genetic material, and the extracellular genetic material is first extracellular genetic material, the process further comprising, receiving, at the fluidic reactor device, a second fluid containing second genetic material, wherein the second fluid is processed through the filter component of the fluidic reactor device, and reacting, at the filter component and using at least the first component of the pump, the first enzyme with the second fluid, wherein the first enzyme is configured to expel the second genetic material from the filter component. The process 1300 may further include reacting, at the second component of the pump, the second enzyme with the second genetic material, wherein the second enzyme is configured to generate second extracellular genetic material from the second genetic material, amplifying the second genetic material to generate amplified second extracellular genetic material, and performing the CRISPR-Cas13 techniques using at least the amplified second extracellular genetic material, wherein the CRISPR-Cas13 techniques are configured to generate the fluorescence based at least in part on the presence of the amplified second extracellular genetic material.

Additionally, or alternatively, the process 1300 may further include wherein the fluid is first fluid, the genetic material is first genetic material, the extracellular genetic material is first extracellular genetic material, the process further comprising receiving, at the fluidic reactor device, a second fluid containing second genetic material, wherein the second fluid is processed through the filter component of the fluidic reactor device, and reacting, at the filter component and using at least the first component of the pump, the first enzyme with the second fluid, wherein the first enzyme is configured to expel the second genetic material from the filter component. The process 1300 may further include reacting, at the second component of the pump, the second enzyme with the second genetic material, wherein the second enzyme is configured to generate second extracellular genetic material from the second genetic material, amplifying the second extracellular genetic material to generate amplified second extracellular genetic material, and performing CRISPR-Cas12 techniques using at least the amplified second extracellular genetic material, wherein the CRISPR-Cas12 techniques are configured to generate the fluorescence based at least in part on the presence of the amplified second extracellular genetic material.

Additionally, or alternatively, the process 1300 may further include wherein detecting fluorescence at the light detection component further comprises emitting a blue light across a solution containing the amplified extracellular genetic material.

Additionally, or alternatively, the process 1300 may further include wherein the genetic material is at least one of single-stranded deoxyribonucleic acid (DNA), double-stranded DNA, single-stranded ribonucleic acid (RNA), or double-stranded RNA.

Additionally, or alternatively, the process 1300 may further include wherein performing the CRISPR-Cas13 techniques comprises introducing at least one of the single-stranded RNA and associated with a fluorophore or the double-stranded RNA and associated with the fluorophore.

Additionally, or alternatively, the process 1300 may further include wherein the genetic material is associated with one of a viral microbe, a prokaryotic organism, and/or a eukaryotic organism.

Additionally, or alternatively, the process 1300 may further include wherein the first component or the second component of the pump is associated with a pressure-driven pump configured to direct the fluid containing the extracellular genetic material to at least one of a component associated with the amplifying and/or a component associated with performing the CRISPR-Cas13 techniques.

Additionally, or alternatively, the process 1300 may further include wherein the first component of the pump is associated with a component configured to store and iteratively load a capsule containing the first enzyme into the first component.

Additionally, or alternatively, the process 1300 may further include wherein at least one of the first component of the pump or the second component of the pump are associated with a stepper motor, the stepper motor being configured to cause one or more magnetic stir bars positioned within the first component of the pump or the second component of the pump to actuate.

Additionally, or alternatively, the process 1300 may further include wherein at least one of the first component of the pump or the second component of the pump are coupled to a stepper motor, the stepper motor being configured to cause a capsule containing the first enzyme or the second enzyme to be introduced into the fluid.

Additionally, or alternatively, the process 1300 may further include transmitting, from a wireless communication device associated with the fluidic reactor device, the data to one or more remote computing devices.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims of the application.

What is claimed is:

1. A method for a fluidic reactor device to detect genetic material, the method comprising:
receiving, at the fluidic reactor device, a fluid containing genetic material, wherein the fluid is processed through a filter component of the fluidic reactor device;
reacting, at the filter component and using at least a first component of a pump, a first enzyme with the fluid, wherein the first enzyme is configured to expel the genetic material from the filter component;
reacting, at a second component of the pump, a second enzyme with the genetic material, wherein the second enzyme is configured to generate extracellular genetic material from the genetic material;
amplifying the extracellular genetic material to generate amplified extracellular genetic material; and
performing CRISPR-Cas13 techniques using at least the amplified extracellular genetic material, wherein the CRISPR-Cas13 techniques are configured to generate fluorescence based at least in part on a presence of the amplified extracellular genetic material.

2. The method of claim 1, wherein the first enzyme is a cellulase enzyme and the second enzyme is a proteinase enzyme.

3. The method of claim 1, wherein reacting the first enzyme with the fluid and reacting the second enzyme with the genetic material comprises an application of at least one of heat or mixing reagents.

4. The method of claim 1, wherein the fluid is first fluid, the genetic material is first genetic material, the extracellular genetic material is first extracellular genetic material, the method further comprising:
receiving, at the fluidic reactor device, a second fluid containing second genetic material, wherein the second fluid is processed through the filter component of the fluidic reactor device;
reacting, at the filter component and using at least the first component of the pump, the first enzyme with the second fluid, wherein the first enzyme is configured to expel the second genetic material from the filter component;
reacting, at the second component of the pump, the second enzyme with the second genetic material, wherein the second enzyme is configured to generate second extracellular genetic material from the second genetic material;
amplifying the second extracellular genetic material to generate amplified second extracellular genetic material; and
performing CRISPR-Cas12 techniques using at least the amplified second extracellular genetic material, wherein the CRISPR-Cas12 techniques are configured to generate the fluorescence based at least in part on the presence of the amplified second extracellular genetic material.

5. The method of claim 1, wherein the genetic material is at least one of single-stranded deoxyribonucleic acid (DNA), double-stranded DNA, single-stranded ribonucleic acid (RNA), or double-stranded RNA.

6. The method of claim 5, wherein performing the CRISPR-Cas13 techniques comprises introducing at least one of the single-stranded RNA and associated with a fluorophore or the double-stranded RNA and associated with the fluorophore.

7. The method of claim 1, wherein the first component or the second component of the pump is associated with a pressure-driven pump configured to direct the fluid containing the extracellular genetic material to at least one of:
a component associated with the amplifying; or
a component associated with performing the CRISPR-Cas13 techniques.

8. The method of claim 1, wherein the first component of the pump is associated with a component configured to store and iteratively load a capsule containing the first enzyme into the first component.

9. The method of claim 1, wherein at least one of the first component of the pump or the second component of the pump is associated with a stepper motor, the stepper motor being configured to cause one or more magnetic stir bars positioned within the first component of the pump or the second component of the pump to actuate.

10. The method of claim 1, wherein at least one of the first component of the pump or the second component of the pump is coupled to a stepper motor, the stepper motor being configured to cause a capsule containing the first enzyme or the second enzyme to be introduced into the fluid.

11. A system comprising:
a filtration component configured to house one or more filters disposed on a platform and one or more gears coupled to the platform, the one or more gears being configured to actuate a rotation of the platform; and
a first reactor including:
a first tube coupled to the filtration component and one or more components of the first reactor, the first reactor including a capsule feeding device configured to load capsules into the one or more components of the first reactor;
one or more actuators coupled to a housing of the one or more components of the first reactor; and
a second tube coupled to the one or more components of the first reactor and a light detection component.

12. The system of claim 11, wherein the capsule feeding device is a first capsule feeding device, the one or more actuators are one or more first actuators, and the light detection component is a first light detection component, the system further comprising:
a second reactor comprising:
a third tube coupled to the filtration component and one or more components of the second reactor, the second reactor including a second capsule feeding device configured to load capsules into the one or more components of the second reactor;
one or more second actuators coupled to a housing of the one or more components of the second reactor; and
a fourth tube coupled to the one or more components of the second reactor and a second light detection component.

13. The system of claim 11, wherein the system further comprises at least one of:
a heating element disposed in the one or more components of the first reactor or the second reactor;
an electrical component disposed in the one or more components of the first reactor or the second reactor; or
a magnetic element coupled to the one or more components of the first reactor or the second reactor.

14. The system of claim 13, wherein the first reactor or the second reactor further comprise a stepper motor configured to actuate a rod that is coupled to the magnetic element.

15. The system of claim 11, wherein the capsule feeding device further comprises a second platform configured to shift across the one or more components of the first reactor or the second reactor.

16. The system of claim 15, wherein the first reactor or the second reactor further comprises a stepper motor configured to actuate a gear, wherein the gear is coupled to the platform.

17. The system of claim 11, wherein the system further comprises a communication device configured to receive instructions from a remote computing device or send data to the remote computing device.

18. The system of claim 11, further comprising a local computing device coupled to the system and configured to store data or communicate the data to a remote computing device.

19. The system of claim 11, further comprising a user interface device coupled to the local computing device and configured to communicate instructions for the system and to the local computing device or the remote computing device.

20. A method for a collecting genetic material, the method comprising:
receiving, at a filter component, a fluid containing genetic material, wherein the filter component at least partially comprises cellulose;
causing a lyophilized cellulase enzyme to expel in a solution, wherein expelling the lyophilized cellulase enzyme in the solution generates a cellulase enzyme;
reacting the cellulase enzyme at the filter component;
causing at least a portion of the filter component comprising the cellulose to degrade; and
causing the genetic material to be expelled from the filter component and into the solution.
